(19) United States
(12) Patent Application Publication
(10) Pub. No.: US 2022/0409525 A1
(43) Pub. Date: Dec. 29, 2022

(54) CELL DELIVERY ARTICLES AND METHODS OF ADMINISTRATION

(71) Applicant: INCUBE LABS, LLC, San Jose, CA (US)

(72) Inventor: Mir A. IMRAN, San Jose, CA (US)

(73) Assignee: INCUBE LABS, LLC, San Jose, CA (US)

(21) Appl. No.: 17/863,551

(22) Filed: Jul. 13, 2022

Related U.S. Application Data
(63) Continuation of application No. PCT/US2021/013210, filed on Jan. 13, 2021.
(60) Provisional application No. 62/960,536, filed on Jan. 13, 2020.
(60) Provisional application No. 63/017,519, filed on Apr. 29, 2020.

(51) Int. Cl. A61K 9/00; C12N 5/071; A61K 35/39; A61K 47/42; A61P 3/10

(52) U.S. Cl. CPC A61K 9/0024; C12N 5/0676; A61K 35/39; A61K 47/42; A61P 3/10

(57) ABSTRACT

This application relates to cell delivery articles and methods for delivering cells into the body in a manner that allows them to incorporate into surrounding tissue and express cell products. The cell delivery articles are generally capable of maintaining viability of the cells for a period of time that allows such incorporation to occur. Additionally, a cell delivery article may include a bio-ghost coating that prevents the cell delivery article from being recognized by the immune system, and/or minimizes or prevents development of fibrotic tissue which can interfere with nutrients and oxygen entering the cell delivery article and reaching the cells. A cell delivery article may be formulated for delivery by various routes of administration.

### CROSS-REFERENCE TO RELATED APPLICATIONS

This application is a continuation of International Application No. PCT/US2021/013210 filed on Jan. 13, 2021 which claims benefit to U.S. Provisional Application Ser. No. 62/960,536 filed Jan. 13, 2020 and U.S. Provisional Application Ser. No. 63/017,519 filed Apr. 29, 2020, the disclosures of which are hereby incorporated in their entirety by reference herein.

### FIELD

This application relates to cell delivery articles and methods for delivering cells into a subject body in a manner that allows the cells to incorporate into surrounding tissue and to express cell products to the tissue. A cell delivery article maintains viability of the cells for a period of time that allows such incorporation to occur. Additionally, a cell delivery article may include a bio-ghost coating that prevents it from being recognized by the immune system, thus keeping it substantially hidden from cells that trigger fibrotic development.

### BACKGROUND

Delivery of cells into a subject body can be useful for treatment of a variety of conditions. Diabetes and pancreatitis are prevalent conditions and are described here by way of example. Many subjects require treatment throughout their lives to control diabetes and/or pancreatitis, undergoing strict diet regimens, glucose monitoring, insulin injections, and dialysis. As the conditions progress, a kidney and/or a pancreas transplant may be needed. For example, the kidney may be transplanted alone, the pancreas may be transplanted alone, the pancreas may be transplanted along with the kidney, or the pancreas may be transplanted in a separate procedure at a time subsequent to a kidney transplant.

The donor kidney and/or pancreas may be transplanted into the pelvic area, and may be transplanted in such a way so as to bypass the liver. The subject's own kidneys and/or pancreas may optionally be removed. There is a high survival rate for both the subjects and the transplanted organs. It has been found that simultaneous pancreas/kidney transplantation, and pancreas transplantation after kidney transplantation, result in improved health of the transplanted kidney over a kidney transplant alone. If a pancreas transplant is successful, glucose levels may be controlled post-transplant for years.

However, one drawback of organ transplantation is the use of immunosuppressant drugs to prevent or reduce rejection of the transplanted organ. Immunosuppressant therapy may begin prior to the transplant procedure, and generally continues for the rest of the subject's life. Although the systemic use of immunosuppressant drugs may be necessary for the success of organ transplantation, these drugs weaken the body's resistance to disease and infection, and are associated with long term side-effects such as osteoporosis and bone marrow suppression. Furthermore, given that organ transplants are invasive procedures, they are inherently associated with surgical risk.

Alternatively to, or additionally to, a whole or partial pancreas organ transplant, pancreatic islet cells have been provided into the liver, such as by an infusion through the liver portal vein. Islet cell infusion into the liver may be performed with or without a pancreatomy. Islet cells include alpha cells, beta cells, and delta cells. Alpha cells produce the hormone glucagon which releases glucose from the liver and fatty acids from fat tissue; beta cells produce insulin; and delta cells produce somatostatin which regulates the endocrine system and affects neurotransmission and cell proliferation. Although islet cell infusion into the liver improves subject outcomes, a diabetic treated with insulin after islet cell liver infusion may become hypoglycemic from the insulin treatment, which may leave a subject intolerant to insulin treatment. Islet cell infusion into the liver also requires an invasive procedure (e.g., trocar placement, catheter positioning through the trocar, and intravenous drip of islets through the catheter).

Pancreatic islet cells have also been disposed within alginate microbeads, and a layer of islet cells have been incorporated into alginate gel sheets; the microbeads or sheets were then implanted in the liver. However, in some cases, this treatment led to thrombosis. Further, an immune response occurred in various cases, such that immune cells (e.g., macrophages) attacked and destroyed the alginate microbeads or sheets and the cells within.

Pancreatic islet cells have also been placed in a pouch of expanded polytetrafluoroethylene (ePTFE) and implanted subcutaneously. However, generally, only the cells near the edges of the pouch would receive sufficient oxygen and nutrients from the body, and the more inner cells within the pouch would die. Moreover, the pouch would suffer fibrotic development (e.g., become covered in a manner similar to a fibrous capsule formation over a joint, or other fibroblast immune or inflammatory response of the body). The islet cells were then not able to receive information from the body and thus could not know what level of glucose was available in the body. Absent such knowledge, the cells would start producing insulin, causing severe problems within the body.

The technologies and treatments for kidney and pancreatic conditions described above suffer many drawbacks, as noted. In addition to diabetes and pancreatitis, other conditions that employ cell therapy have encountered obstacles, preventing them from becoming widely used. For example, conditions such as cancer, an autoimmune disease, and a neurological disorder may include cell therapy as part of a treatment plan. However, the ability to maintain an environment that preserves cell viability has been challenging. Furthermore, in many treatments, the cells are administered intravenously, requiring a visit to a hospital or clinic, which is inconvenient, especially if several doses need to be administered.

Given the challenges faced by present cell delivery treatments and techniques, it would be beneficial to have a way to deliver cells in a more convenient fashion, with minimal invasiveness or without invasive procedures, without using systemic immunosuppressive treatment, and in a manner that maintains cell viability until and after the cells reach their target site.

### SUMMARY

Described herein are cell delivery articles for delivering various types of cells into a body. The cell delivery articles may be delivered in a manner that allows them to incorporate into surrounding tissues and express cell products. Additionally, the cell delivery articles are capable of supporting the cells by providing nutrients and oxygen for a period of time to allow such incorporation to occur. A cell delivery article may further include a bio-ghost coating that prevents it from being recognized by the immune system, and/or minimizes or prevents development of fibrotic tissue from interfering with nutrients and oxygen entering the cell delivery article and reaching the cells. Methods for delivering the cell delivery articles to tissue sites and methods of administering the cell delivery articles are also described herein.

Methods of delivering cells to a tissue site are also described herein that generally include: 1) introducing a cell delivery article into the body of a subject, where the cell delivery article includes cells within a reservoir; 2) maintaining or supporting the cells in a medium disposed within the reservoir and with oxygen generated in the cell delivery article; and 3) preventing recognition of at least a portion of the cell delivery article by the immune system of the subject using a bio-ghost coating. The methods may further include implanting, attaching, or retaining the cell delivery article at a tissue site using a delivery assist mechanism. After or simultaneously with incorporation, the methods may include expression of a cell product by the cells.

Introduction of the cell delivery article into the body of the subject may be accomplished orally or parenterally. Parenteral delivery may include, for example, intravenous, intramuscular, subcutaneous, intraperitoneal, intrathecal, intraocular, and intra-articular routes. The cell delivery article may also be introduced by topical application to the skin, a mucosal surface, or a surface of a burn or wound.

Further described herein are methods of administering cells to a subject. The methods generally include: 1) providing a treatment regimen for a condition, the treatment regimen including a dosing schedule; and 2) administering a dose, the dose including one or more cell delivery articles in a dosage form according to the dosing schedule. Here the one or more cell delivery articles include cells, and a medium and an oxygen supply for supporting the cells. The cell delivery articles are provided in a dosage form suitable for the intended route of delivery.

### BRIEF DESCRIPTION OF THE DRAWINGS

FIG. 1A, FIG. 1B, FIG. 1C, and FIG. 1D illustrate embodiments of a cell delivery article.

FIG. 2 illustrates an embodiment of a cell delivery article.

FIG. 3 illustrates an embodiment of a cell delivery article.

FIG. 4A, FIG. 4B, FIG. 4C, and FIG. 4D illustrate embodiments of a cell delivery article.

FIG. 5 illustrates an embodiment of a cell delivery article and method of manufacture.

FIG. 6 illustrates an embodiment of a cell delivery article and method of manufacture.

FIG. 7A and FIG. 7B illustrate embodiments of a cell delivery article including a delivery assist mechanism.

FIG. 8A and FIG. 8B illustrate embodiments of delivery assist mechanisms disposed on a cell delivery article.

FIG. 9A and FIG. 9B illustrate embodiments of delivery assist mechanisms disposed on a cell delivery article.

FIG. 10 illustrates an embodiment of a bio-ghost coating disposed on a cell delivery article.

FIG. 11 illustrates an embodiment of a bio-ghost coating disposed on an outer wall of a reservoir of a cell delivery article.

FIG. 12 illustrates an embodiment of a bio-ghost coating disposed on a reservoir to be positioned in a cell delivery article.

FIG. 13 illustrates an embodiment of a cell delivery article.

FIG. 14 illustrates the P-15 cell binding domain on collagen fibers.

FIG. 15A and FIG. 15B are scanning electron micrographs (SEMs) that compare migration of cells on membranes with and without a biomimetic coating.

FIG. 16 illustrates an embodiment of a shell.

FIG. 17A illustrates an embodiment of a plug disposed in a shell.

FIG. 17B illustrates an embodiment of a plug disposed in a shell.

FIG. 18 illustrates an embodiment of a membrane tube as it is coated with a bio-ghost material.

FIG. 19 illustrates an embodiment of a membrane tube disposed in a shell.

FIG. 20A illustrates an embodiment of a gel medium disposed in a membrane tube.

FIG. 20B illustrates an embodiment of a gel medium mixed in a membrane tube.

FIG. 21 illustrates an embodiment of two plugs and a membrane tube in a shell.

FIG. 22 illustrates an embodiment of a sealed cell delivery article.

FIG. 23 illustrates an embodiment of a sealed cell delivery article disposed in a sealed chamber.

FIG. 24A and FIG. 24B illustrate an embodiment of a transporter for a cell delivery article.

### DETAILED DESCRIPTION

When used in the present disclosure, the terms “e.g.,” “such as”, “for example”, “for an example”, “for another example”, “examples of”, “by way of example”, and “etc.” indicates that a list of one or more non-limiting example(s) precedes or follows; it is to be understood that other examples not listed are also within the scope of the present disclosure.

As used herein, the singular terms “a,” “an,” and “the” may include plural referents unless the context clearly dictates otherwise. Reference to an object in the singular is not intended to mean “one and only one” unless explicitly so stated, but rather “one or more.”

As used herein, the term “set” refers to a collection of one or more objects. Thus, for example, a set of objects can include a single object or multiple objects.

The term “in an embodiment” or a variation thereof (e.g., “in another embodiment” or “in one embodiment”) refers herein to use in at least one embodiment, and in no case limits the scope of the present disclosure to only the embodiments described. Accordingly, components each described in separate embodiments can be used together in a single embodiment without explicitly being described as being used together, and a component described in one embodiment can be incorporated into another embodiment without explicitly being described as being used together.

The term “component” refers herein to one item of a set of one or more items that together make up a device or formulation under discussion. A component may be in a solid, powder, gel, plasma, fluid, gas, or other form. For example, a device may include multiple solid components which are assembled together to structure the device and may further include a gel component that is disposed in the device. For another example, a formulation may include two or more powdered and/or fluid components which are mixed together to make the formulation. The term “framing portion” refers herein to one or more components which define a shape of a device, such as a shape of a cell delivery article or a shape of a plug. The framing portion may change structure after manufacture, such that the shape of the device changes.

The term “design” or a grammatical variation thereof (e.g., “designing” and “designed”) refers herein to characteristics intentionally incorporated into a design based on estimates of tolerances related to the design (e.g., component tolerances and/or manufacturing tolerances) and estimates of environmental conditions expected to be encountered by the design (e.g., temperature, humidity, external or internal ambient pressure, external or internal mechanical pressure or stress, age of product, physiology, body chemistry, biological composition and/or chemical compositions of fluids and tissue, pH, species, diet, health, gender, age, ancestry, disease, tissue damage, shelf life, or the combination of such); it is to be understood that actual tolerances and environmental conditions before and/or after delivery can affect such designed characteristics so that different components or devices with a same design can have different actual values with respect to those designed characteristics. Design encompasses also variations or modifications to the design, a component or device structured in accordance with the design, and design modifications implemented on a component or device after it is manufactured.

The term “manufacture” or a grammatical variation thereof (e.g., “manufacturing” and “manufactured”) as related to a component or device refers herein to making the component or device, whether made wholly or in part by hand or made wholly or in part in an automated fashion.

The term “structured” or a grammatical variation thereof (e.g., “structure” or “structuring”) refers herein to a component or device that is manufactured according to a concept or design or variations thereof or modifications thereto (whether such variations or modifications occur before, during, or after manufacture) whether or not such concept or design is captured in a writing.

The term “body” refers herein to a member of any life-form domain or non-life-form domain Some examples herein refer to animalia anatomy and conditions for convenience, without limiting the scope of the subjects to which a cell delivery article in accordance with the present disclosure may be applicable.

The term “subject” refers herein to a body into which a cell delivery article is, or is intended to be, positioned. By way of a few examples: with respect to humans, a subject may be a patient under the treatment of a health care professional; with respect to flora, a subject may be a plant; with respect to bacteria, a subject may be a bacterial colony; with respect to non-life-forms, a subject may be an oil spill or waste treatment sludge. Other examples are within the scope of the present disclosure.

The term “tissue site” refers herein to any location within a body at a site where a cell delivery article is positioned or is intended to be positioned (e.g., tissue of the peritoneum, heart, liver, gastrointestinal (GI) tract, eye, brain, skin, another organ, subcutaneous tissue, interstitial tissue, connective tissue, or other portion of an animalia body). A cell delivery article may be structured for positioning at a particular tissue site, may be delivered to a tissue site for which it was structured, delivered to a tissue site for which it was not structured, or inadvertently delivered to an unintended tissue site. The tissue site refers to the designed for or intended tissue site prior to positioning as well as the present tissue site after positioning; if the cell delivery article migrates from an initial tissue site, tissue at its present location along the migratory path is also referred to as the tissue site. The cell delivery article remains at a tissue site until it degrades and/or is expelled from the body.

The term “biological matter” refers herein to blood, tissue, fluid, enzymes, and other secretions of a body. The term “digestive matter” refers herein to biological matter along the GI tract in an animalia body, and other matter (e.g., food in an undigested or a digested form) traversing the GI tract.

The term “ingest” or a grammatical variation thereof (e.g., “ingesting” and “ingested”) refers herein to taking into the stomach, whether by swallowing or by other means of depositing into the stomach (e.g., by depositing into the stomach by endoscope or depositing into the stomach via a port).

The term “fluid” refers herein to a liquid, and encompasses moisture and humidity. The term “fluidic environment” refers herein to an environment in which one or more fluids are present. In an embodiment, a cell delivery article in accordance with the present disclosure is structured to be disposed within a body, and thus biological matter or digestive matter results in a fluidic environment.

The term “degrade” or a grammatical variation thereof (e.g., “degrading”, “degraded”, and “degradation”) refers herein to weakening, partially degrading, or fully degrading, such as by dissolution, chemical degradation (including biodegradation), decomposition, chemical modification, mechanical degradation, or disintegration, which encompasses also, without limitation, dissolving, crumbling, deforming, shriveling, or shrinking. The term “non-degradable” refers to an expectation that degradation will be minimal, or within a certain acceptable design percentage, for at least an expected duration in an expected environment.

The term “degradation rate” or a grammatical variation thereof (e.g., “rate of degradation”) refers herein to a rate at which a material degrades. A designed degradation rate of a material in a particular implementation can be defined by a rate at which the material is expected to degrade under expected conditions (e.g., in physiological conditions) at a target tissue site. A designed degradation time for a particular implementation can refer to a designed time to complete degradation or a designed time to a partial degradation sufficient to accomplish a design purpose (e.g., breach). Accordingly, a designed degradation time can be specific to a cell delivery article and/or specific to expected conditions at a target tissue site. A designed degradation time can be short or long and can be defined in terms of approximate times, maximum times, or minimum times. For example, a designed degradation time for a component can be about 1 minute, less than 1 minute, greater than 1 minute, about 5 minutes, less than 5 minutes, greater than 5 minutes, about 30 minutes, less than 30 minutes, greater than 30 minutes, and so forth with respect to minutes; or about 1 hour, less than 1 hour, greater than 1 hour, about 2 hours, less than 2 hours, greater than 2 hours, and so forth with respect to hours; or about 1 day, less than 1 day, greater than 1 day, about 1.5 days, less than 1.5 days, greater than 1.5 days, about 2 days, less than 2 days, greater than 2 days, and so forth with respect to days; or about 1 week, less than 1 week, greater than 1 week, about 2 weeks, less than 2 weeks, greater than 2 weeks, about 3 weeks, less than 3 weeks, greater than 3 weeks, and so forth with respect to weeks; or about 1 month, less than 1 month, greater than 1 month, about 2 months, less than 2 months, greater than 2 months, about 6 months, less than 6 months, greater than 6 months, and so forth with respect to months; or about 1 year, less than 1 year, greater than 1 year, about 2 years, less than 2 years, greater than 2 years, about 5 years, less than 5 years, greater than 5 years, about 10 years, less than 10 years, greater than 10 years, and so forth with respect to years; or other designed degradation approximate time, minimum time, or maximum time. A designed degradation time can be defined in terms of a limited range. For example, a designed degradation time can be in terms of a range of about 12-24 hours, about 1-6 months, about 1-2 years, or other range.

The terms “substantially” and “about” are used herein to describe and account for small variations. For example, when used in conjunction with a numerical value, the terms can refer to a variation in the value of less than or equal to ±10%, such as less than or equal to ±5%, less than or equal to ±4%, less than or equal to ±3%, less than or equal to ±2%, less than or equal to ±1%, less than or equal to ±0.5%, less than or equal to ±0.1%, or less than or equal to ±0.05%.

As used herein, a range of numbers includes any number within the range, or any sub-range if the minimum and maximum numbers in the sub-range fall within the range. Thus, for example, “<9” can refer to any number less than nine, or any sub-range of numbers where the minimum of the sub-range is greater than or equal to zero and the maximum of the sub-range is less than nine.

Amounts, ratios, and other numerical values may sometimes be presented herein in a range format. It is to be understood that such range format is used for convenience and brevity and should be understood flexibly to include numerical values explicitly specified as limits of a range, but also to include all individual numerical values or sub-ranges encompassed within that range as if each numerical value and sub-range is explicitly specified. For example, a ratio in the range of about 1 to about 200 should be understood to include the explicitly recited limits of about 1 and about 200, but also to include individual ratios such as about 2, about 3, and about 4, and sub-ranges such as about 10 to about 50, about 20 to about 100, and so forth.

Certain measurement units used may be abbreviated herein as follows: nanometers (“nm”), micrometers (“μm”), millimeters (“mm”), and centimeters (“cm”).

The cell delivery articles described herein may function as a platform for delivering various types of cells into a body. The cell delivery articles may be delivered in a manner that allows the cells to incorporate into surrounding tissues and express cell products. The cells and/or their cell products may be used to treat a condition or disease. Additionally, the cell delivery articles are generally capable of maintaining cell viability for a period of time. A cell delivery article may further include a coating that prevents the cell delivery article from being recognized by the immune system, and/or minimizes or prevents fibrotic development from interfering with nutrients and oxygen entering the cell delivery article and reaching the cells. Methods for delivering the cell delivery articles to tissue sites and methods of administering the cell delivery articles are also described herein.

Among other advantages and benefits that will be apparent from the figures and discussion of the present disclosure, embodiments of cell delivery articles encompassed by the present disclosure provide one or more of the following advantages and benefits:
A cell delivery article contains an oxygen supply to maintain viability of cells within the cell delivery article after manufacture of the cell delivery article and at least until incorporation of the cells into a tissue site.
Immunosuppressant therapy is not needed before, during, or after the delivery of a cell delivery article. Accordingly, cell delivery articles may be used in treatments for a broad range of conditions, and in a broad range of subjects for whom it is desirable to avoid immunosuppressant therapy.
A cell delivery article includes a bioactive, bioinactive, bioinert, or bioresponsive coating that renders coated portions of the cell delivery article effectively invisible to the immune system (each, and any, such coating is referred to herein as a “bio-ghost” coating, and the action of hiding a portion of the cell delivery article using a bio-ghost coating is referred to herein as “bio-ghosting”). For example, a bio-ghost coating can include a bioactive material that elicits a response from living tissue, organisms or cells, a bioinductive material that induces a response in a biological system, a biomaterial that interacts with a biological system, or a biomimetic material that mimics natural structures of the body.
A cell delivery article avoids development of fibrosis that could cover the cell delivery article and block the cells in the cell delivery article from receiving from the body a supply of oxygen and nutrients. In an embodiment, a bio-ghost coating is disposed on a cell delivery article to avoid fibrotic response.
A cell delivery article attracts body cells from a tissue site of a body to bond to a bio-ghost coating so that the cell delivery article appears to the immune system of the body as being part of the body. The body cells can then provide oxygen and nutrients to the cells contained in the cell delivery article.
A cell delivery article avoids triggering an immune response by the body against cells contained within the cell delivery article.
Interstitial fluid is able to reach cells within a cell delivery article. For example, in an embodiment in which a cell delivery article includes islet cells, the islet cells can determine a glucose level in the body and respond appropriately.
Oxygen and nutrients reach a majority of the cells within a cell delivery article (e.g., rather than being limited to reaching cells on the edge as in the case of a pouch).
The implanted cells are perfused with sufficient oxygen and nutrients so that they remain viable between the time the cells are separated from the donor and the time the cells are placed in the subject body.

### CELL DELIVERY ARTICLES

A cell delivery article as described herein is structured to define a cavity. A reservoir is disposed or defined within the cavity, and cells are disposed within the reservoir. A plug is disposed within the cavity and is in chemical communication with the reservoir. The plug may include an oxygen supply that is structured to aid in supporting the cells for a time before the cell delivery article is positioned at a tissue site in a body and, in some embodiments, for a time after the cell delivery article is positioned at the tissue site. A medium may be disposed within the reservoir. The medium may be structured to aid in supporting the cells physically, such as by providing a cushioning environment to protect the cells from damage caused by movement, and/or such as by providing nutrients and/or other substances to maintain the cells in a viable state; the medium may additionally or alternatively include a substance that minimizes or prevents an immune response to the cells by the body. A delivery assist mechanism may be provided that is structured to help implant, attach, and/or retain the cell delivery article at the tissue site. A bio-ghost coating may entirely or partially cover the cell delivery article, the bio-ghost coating being structured to minimize or prevent an immune response to the cell delivery article or portions thereof by the body.

A cell delivery article may have any suitable size and shape. These characteristics may depend on such factors as the intended tissue site for delivery, the dose of cells (e.g., number, weight, or volume of cells) to be delivered per cell delivery article, and the route of administration. A cross-sectional dimension of a cell delivery article may range from about 1 μm to about 1000 μm, and a length of a cell delivery article may range from about 2 μm to about 50 cm. In an embodiment, a diameter of a cell delivery article is about 1.7 μm, and a lengthwise measurement of the cell delivery article is about 5.5 μm. A cross-sectional shape of a cell delivery article may be substantially circular or other elliptical shape, or substantially a rectangle or triangle or other polygonal shape, or the cross-sectional shape may be irregular. The cross-sectional shape and circumference may vary along a length of a cell delivery article.

### Reservoir

The reservoir included in a cell delivery article includes a reservoir outer wall. In an embodiment, a wall of the cell delivery article constitutes the reservoir outer wall, such that the wall of the cell delivery article defines an outer dimension of the reservoir. In an embodiment, the reservoir outer wall is separate from other walls of the cell delivery article.

The reservoir outer wall may have a similar cross-sectional shape and circumference to a wall of a remainder of the cell delivery article, or may be different from walls of a remainder of the cell delivery article in cross-sectional shape and/or circumference. In an embodiment, a circumference of the reservoir outer wall is less than a circumference of a wall of a remainder of the cell delivery article, such that when a coating (e.g., a bio-ghost coating) is disposed over the reservoir outer wall, the circumference of the reservoir outer wall is approximately the same as the circumference of the wall of the remainder of the cell delivery article.

The reservoir outer wall may be rigid, semi-rigid, flexible, or a combination of the foregoing. For example, in a treatment location in which the cell delivery article may encounter forces against it, the reservoir outer wall may be structured to be semi-rigid to rigid such that the reservoir outer wall protects cells in the reservoir from damage. For another example, a reservoir outer wall for a largely fluidic environment not expected to exert significant forces against the cell delivery article may have flexible walls.

In an embodiment, a cell delivery article includes a reservoir outer wall that has a rigid or semi-rigid framing portion around the reservoir, with a flexible covering over the framing portion, under the framing portion, or interspersed within the framing portion.

In general, a design of a framing portion and a material selection for the cell delivery article and its reservoir define the rigidity of the reservoir outer wall. Additionally, manufacturing process capabilities may lead to a selection of material(s) which result in a reservoir outer wall that is more rigid or less rigid.

The reservoir outer wall is porous. For example, pores in the reservoir outer wall may be sized to allow passage of water (e.g., water present in biological matter or digestive matter at the tissue site), oxygen, nutrients, and other cell viability factors into the reservoir, while blocking passage of immune cells into the reservoir. In an embodiment, pores in the reservoir outer wall have a diameter greater than about 0.1 nm to allow cell viability factors to pass into the reservoir from the body. In an embodiment, pores in the reservoir outer wall have a diameter less than about 10 μm to block neutrophils, eosinophils, basophils, large lymphocytes, and monocytes from entering the reservoir from the body, or less than about 7 μm to additionally block small lymphocytes.

In an embodiment, pores in the reservoir outer wall are sized to allow cell products expressed by the cells to pass from the reservoir into the body. In an embodiment in which islets are contained in the reservoir, pores in the reservoir outer wall may have a diameter of greater than about 0.2 μm, to allow glucagon, insulin, and somatostatin produced by the islets to pass from the reservoir into the body.

In an embodiment, the reservoir outer wall is, or includes, a membrane. In an embodiment, leachable nanoparticles of sodium or potassium chloride are added to a membrane, then the membrane is soaked in water to remove salts, leaving pores in the membrane. In an embodiment, a membrane is formed in a tubular structure.

In an embodiment, regardless of pore size, the reservoir outer wall allows unidirectional flow of substances undesirable within the reservoir (e.g., immune cells, viruses, bacteria, or the like) such that the undesirable substances if present can flow out of the reservoir but not into the reservoir. In an embodiment, the reservoir outer wall includes an antimicrobial, antiviral, and/or anti-immunosuppressant membrane or coating.

The pores may be arranged in any suitable fashion on the reservoir outer wall. In an embodiment, the pores are homogeneously spaced throughout the reservoir outer wall. In an embodiment, the pores are arranged in a pattern on the reservoir outer wall. A pattern may include groups of pores that are symmetrically or asymmetrically spaced apart along the reservoir outer wall. A pattern may include pores with a first dimension (e.g., diameter) in one or more areas of the reservoir outer wall and pores with a second smaller dimension (e.g., diameter) in other areas of the reservoir outer wall, such as to limit flow of larger molecules to a particular portion of the reservoir outer wall.

The reservoir outer wall may be formed in whole or in part from a biocompatible material, for example, a biocompatible polymer or a biocompatible metal.

In an embodiment, the material(s) used to form the reservoir outer wall are generally degradable, and structured to degrade at a rate that provides the reservoir outer wall until a time after the cell delivery article is positioned at a tissue site. In an embodiment, a cell delivery article is effectively an autograft: the cell delivery article contains autologous cells (from a subject's own body or cells cloned or copied from cells from the subject's own body); after the cells are incorporated into the tissue site, framing portions of the cell delivery article (including the reservoir outer wall) may not be needed to protect the autologous cells from an immunosuppressive response, and the framing portions may be structured to degrade quickly after reaching the tissue site (e.g., within seconds, minutes, hours, days, or weeks).

In an embodiment, the material(s) used to form the reservoir outer wall are generally not degradable. In an embodiment, a cell delivery article is effectively an allograft: the cell delivery article contains allogeneic cells (from a donor body); after the cells are incorporated into the tissue site, framing portions of the cell delivery article (including the reservoir outer wall) may be needed to protect the allogeneic cells from an immunosuppressive response, such that the framing portions may be structured to not degrade for an extended time after reaching the tissue site (e.g., after weeks, months, or years).

In an embodiment, the reservoir outer wall is formed from both degradable and non-degradable material(s), and the reservoir outer wall is structured such that portions of the reservoir outer wall degrade rapidly and portions degrade more slowly or do not significantly degrade. In an embodiment, the reservoir outer wall is formed with an outer layer of one or more degradable materials that degrade within seconds or minutes after deployment at a tissue site, and an inner layer of one or more non-degradable materials that resist degradation and thus maintain a reservoir outer wall for an extended time.

A standardized cell delivery article intended to be used for multiple therapy types may be structured to degrade within a time frame suitable for all of the multiple therapy types, such as being structured to degrade after a time period that is the longest time period needed for any of the therapies. For example, a standardized cell delivery article may be structured to degrade after a time period suitable for an allograft, and may also be used to deliver autologous cells.

Some examples of materials that may be used to form the reservoir outer wall include, without limitation, polytetrafluoroethylene (PTFE), ePTFE, polyimide, polysulfone, cellulose, polylactic acid (PLA), poly(glycolic acid) (PGA), or a combination of PLA and PGA (e.g., PLGA or PGLA). In an embodiment, the reservoir outer wall is, or includes, a porous polyimide.

### Coating

The reservoir outer wall may include a bio-ghost coating or bio-ghost treatment, either of which is referred to herein as a bio-ghost coating for convenience and without limitation. A bio-ghost coating is structured to prevent triggering an immune response in a subject receiving the cell delivery article. In an embodiment, a bio-ghost coating includes a poly-1-arginine-based biomaterial. In an embodiment, a bio-ghost coating includes a biomimetic peptide. The biomimetic peptide may be a multi-arm peptide that is an analogue of the cell binding domain of collagen. In an embodiment, the biomimetic peptide is a P-15 peptide. In an embodiment, a bio-ghost coating includes a biomimetic calcium phosphate (Ca—P), hydroxyapatite/tricalcium phosphate (HAp), nanoparticle network of crystalline HAp, gas plasma, other bio-ghost coating, or a combination of the foregoing.

A bio-ghost coating provides a capability to forego treating a subject with immunosuppressants while still delivering cell therapy into a body. Immunosuppressants are often undesirable as they can leave a subject with an increased susceptibility to disease. Accordingly, cell therapy may be provided for a broad range of conditions and to a much broader set of subjects using cell delivery articles according to the present disclosure than would be the case for other therapies (including organ transplants) for which an immunosuppressant is required.

### Medium

A medium within the reservoir is structured to support the cells until the cell delivery article is administered to the subject, reaches its intended tissue site, and the cells contained in the reservoir of the cell delivery article are partially or entirely incorporated into the tissue site. As used in this document, the term “support the cells” or a grammatical variation thereof (e.g., “the cells are supported”, “supporting the cells”, or “supported the cells”) refers to providing a physical medium to provide one or more of the following services: protect the cells from damage (e.g., during article manufacture, shipping, handling, or delivery); protect the cells from immunologic attack; provide nutrients, oxygen, or other cell viability factors to the cells; provide water from the medium to combine with an oxygen supply component in a plug of the cell delivery article; receive water from a tissue site to replenish water stores in the medium for combining with an oxygen supply component in a plug of the cell delivery article; receive oxygen and nutrients from a tissue site to provide the oxygen and nutrients to the cells; receive cell products from the cells to provide to the tissue site; or other service as applicable.

The phrase “incorporated into the tissue site” or a grammatical variation thereof refers herein to a state in which at least some of the cells in a cell delivery article are being sustained by oxygen, nutrients, and/or other cell viability factors from the body at the tissue site.

In an embodiment, when cells are incorporated into a tissue site, the cells have been released from the cell delivery article into the body at the tissue site, such as by degradation of the cell delivery article or degradation of at least a portion of a reservoir outer wall of the cell delivery article, and the cells are at least partially dispersed into the tissue site and maintained in a viable state by the tissue site. For example, autologous cells may be released from the cell delivery article. In an embodiment, allogeneic cells may be released from the cell delivery article, such as when an immune response is desired to be triggered by release of the allogeneic cells, or when the cells are immune cells.

In an embodiment, when the cells are incorporated into the tissue site, the cells have been retained within the cell delivery article and are sustained by oxygen and nutrients entering the cell delivery article from the tissue site through the pores of the reservoir outer wall.

A medium may continue to react with an oxygen supply component in a plug of a cell delivery article, and/or may continue to provide nutrients and/or other cell factors to cells in the cell delivery article, after the cells are incorporated into a tissue site.

A time period for support of cells by a medium, and/or providing oxygen to the cells in concert with an oxygen supply component in a plug of a cell delivery article, may range from about 24 hours to about 24 weeks (about 6 months), or longer. For example, the medium may be structured to at least partially continue to support the cells for about 24 hours, for about 2 days, for about 3 days, for about 4 days, for about 5 days, for about 6 days, for about one week, for about two weeks, for about three weeks, for about four weeks, for about five weeks, for about six weeks, for about seven weeks, for about 2 months, for about 3 months, for about 4 months, for about 5 months, for about 6 months, and so forth. In an embodiment, the cell delivery article (including the medium and the oxygen supply component) is structured to at least partially support the cells for one or more years after incorporation into a tissue site.

As noted above, a medium may include nutrients and other cell viability factors in a carrier substance, and/or a medium can provide resistance to immunosuppressant attacks by the body against the cells in the reservoir. Mediums may include, for example, such nutrients as choline, folic acid, nicotinamide, pantothenic acid, pyridoxal, riboflavin, thiamine, and inositol, among others. Nutrients may be provided in carrier substances such as alginate, alginate gel, polylysine, PLL/poly-L-ornithine, agarose, polyethylene glycol (PEG), chitosan, collagen, polydiallydimethyl ammonium chloride, another substance, or combinations of the foregoing.

In an embodiment, a reservoir of a cell delivery article is structured to contain up to 1000 cells.

A dosage number of cells in a cell delivery article may be selected according to a treatment plan; such as more than 200, between 100 and 300, less than 500, and so forth in any number or in any range suitable for the cell delivery article structure.

### Plug

A cell delivery article as described herein generally includes at least one plug disposed in a cavity defined by the cell delivery article. A cell delivery article may include at least one plug, at least two plugs, at least three plugs, at least four plugs, at least five plugs, and so forth. The number of plugs included may depend, for example, on a size and/or shape of the cell delivery article, a size and/or shape of a cavity defined by the cell delivery article, a size and/or shape of the plug(s), a number of cells within a reservoir disposed in the cell delivery article, and/or a period of time that the cells in the cell delivery article are estimated to require support to maintain their viability. In an embodiment, a cell delivery article includes two plugs. In an embodiment, a cell delivery article includes one plug.

In an embodiment, one or more plugs and a reservoir are contained in a cavity of a cell delivery article.

In an embodiment, a cell delivery article with one or more plug(s) together define a volume in the cavity, and that volume is the reservoir.

In an embodiment, a cell delivery article defines multiple cavities, and one or more plugs are disposed in one or more of the cavities. In an embodiment, a cell delivery article includes multiple reservoirs and one or more plugs; each reservoir is in chemical communication with at least one plug.

Each plug includes a material forming a framing portion of the plug, and an oxygen supply component. In an embodiment, a plug includes biocompatible material.

In an embodiment, a material forming a framing portion of a plug includes any suitable polymer. For example, a percentage by weight of a polymer in a plug that includes the polymer and an oxygen supply component may be about 20% to about 80%. Other percentages may be employed depending on an estimated time period for the cell delivery article to reach the tissue site, an estimated time period for the cells to be incorporated into the tissue site, a type of cell being delivered, a number of cells disposed within the cell delivery article, a storage temperate of the cell delivery article prior to delivery, and/or other considerations. Additionally, a weight percent of polymer to oxygen supply component in a plug can be based at least in part on a transmission rate of water into and through the polymer, and/or a transmission rate of oxygen out of the polymer.

In an embodiment, a plug is made of a polysiloxane (silicone), a polysulfone, polyurethane, a nylon, another polymer, or a combination of the foregoing. The material(s) of the plug are selected for a desired transmission rate of water from a selected medium into and through the plug, and a desired transmission rate of oxygen through and out of the plug and into the medium. For example, material(s) of a plug of a cell delivery article which is structured to be maintained at a low temperature prior to delivery to a body may be selected for a lower transmission rate as compared to material(s) used in a plug of a cell delivery article which will be maintained at a higher temperature, because the cells in the reservoir may require less oxygen at lower temperatures. In an embodiment, polymers are cross-linked to achieve material properties of a plug which increase or decrease a rate of transmission of water and/or oxygen.

In an embodiment, an oxygen supply component is activated to generate oxygen by reacting it with water from a medium. For example, the oxygen supply component may be, or may include, calcium peroxide, sodium peroxide, or magnesium peroxide, other oxidic component, or a combination of two or more of the foregoing. Water molecules in the medium combine with the oxygen supply component to form oxygen, which traverses the medium to oxygenate cells in the medium. After deployment of the cell delivery article at a tissue site, the reservoir outer wall may allow water to pass into the reservoir from biological matter at the tissue site, such as to continue combining the oxygen supply component with water for conversion into oxygen for the cells until the oxygen supply component is depleted. After incorporation into the tissue site, the cells may receive oxygen from the tissue site through the reservoir outer wall and the medium, additionally or alternatively to receiving oxygen from the oxygen supply component.

In an embodiment, the oxygen supply component in a plug is calcium peroxide, permeated throughout a mass of silicone which is a framing portion of the plug.

### Delivery Assist Mechanism

A cell delivery article as described herein may be structured to include a delivery assist mechanism. The delivery assist mechanism can be structured to promote movement of the cell delivery article into and/or through tissue, and/or to aid in retention of the cell delivery article at an intended tissue site. In an embodiment, the delivery assist mechanism is structured to degrade after a selected time period, such as after delivery of the cell delivery article to a tissue site, or after a time period sufficient to allow cells in the cell delivery article to incorporate into the tissue site.

In an embodiment, a cell delivery article is structured to be delivered through tissue, and the delivery assist mechanism is structured to withstand degradation at least until the cell delivery article has been delivered. For example, an expulsion force may be used to expel a cell delivery article from a dosage form so that the cell delivery article passes through tissue and is deployed within a body; a delivery assist mechanism may be structured to begin degrading starting from the moment the cell delivery article is expelled from the dosage form, while withstanding significant degradation for a time period at least sufficient to pass through tissue (e.g., in terms of microseconds, milliseconds, seconds, minutes, or longer). For another example, a cell delivery article may be placed into a body manually, such as by using a trocar, catheter, needle, forceps, or other placement tool to position and release the cell delivery article; a delivery assist mechanism may be structured to begin degrading starting from the moment the cell delivery article begins its route to placement, while withstanding significant degradation for a time period sufficient to reach deployment (e.g., in terms of milliseconds, seconds, minutes, or longer). For a further example, a dosage form may be placed manually into a body (e.g., by trocar, catheter, needle, forceps, or other placement mechanism) and the dosage form is activated manually or is self-activated to expel a cell delivery article into tissue; a delivery assist mechanism may be structured to begin degrading starting from the moment the cell delivery article is expelled from the dosage form, while withstanding significant degradation for a time period sufficient to reach a resting position (e.g., in terms of microseconds, milliseconds, second, minutes, or more).

A delivery assist mechanism may include a tapered end to promote movement of the cell delivery article into and/or through tissue. The taper may be symmetrical about an axis of the tapered end, or may be asymmetrical along the axis. In an embodiment, the delivery assist mechanism is conical. A delivery assist mechanism may be structured with a sharp tip, which, in an embodiment, is a separate component added to the delivery assist mechanism during manufacture.

A delivery assist mechanism (including a tip if applicable) may be made from any suitable material(s). In an embodiment, the delivery assist mechanism includes biocompatible materials. In an embodiment, a delivery assist mechanism is formed from polyethylene oxide. In an embodiment, a delivery assist mechanism includes a tip formed from magnesium (e.g., stamped or etched from a sheet of magnesium, or molded from magnesium into a desired shape).

Additionally or alternatively to promoting movement of a cell delivery article into and/or through tissue, a delivery assist mechanism may be structured to promote retention of a cell delivery article within tissue in treatments where the cell delivery article is structured to be maintained at, or in close proximity to, an initial tissue site. In an embodiment, a cell delivery article includes one or more protrusions which serve to resist (e.g., restrict, minimize, prevent, block, etc.) movement of the cell delivery article once deployed. Such protrusions may be structured for short-term resistance or long-term resistance to movement. In an embodiment, a cell delivery article is structured with one or more protrusions reminiscent of fish scales, where each protrusion resists movement in one direction; multiple such protrusions may face in different directions to collectively resist movement in multiple directions. In an embodiment, a cell delivery article is structured with one or more protrusions having hooked or barbed portions, where each protrusion generally resists movement away from tissue in which the hooked or barbed portion is engaged. In an embodiment, a cell delivery article is structured with one or more protrusions shaped to promote tissue growth around the protrusion; for example, a protrusion may be a flap with holes so that tissue may grow in and around the flap, or a protrusion may have a helical shape so that tissue may grow around the helix. Other shapes of protrusions to resist movement are also encompassed by the present disclosure. A protrusion may resist movement in the short term, the long term, or both in the short term and the long term. For example, each of the foregoing described protrusions may have a short-term effect and a long-term effect. A cell delivery article may be structured with multiple protrusions of varying design to provide sufficient short-term and long-term resistance to movement. In an embodiment, a cell delivery article includes protrusions which are structured for short-term resistance and are degradable (e.g., over days, weeks, or months), and the cell delivery article also includes protrusions which are structured for long-term resistance and are not degradable; once the short-term protrusions are degraded at a tissue site and expelled from the body, there are fewer foreign materials left remaining in the body and the long-term protrusions suffice to hold the cell delivery article in place.

In an embodiment of a cell delivery article structured to include one or more delivery assist mechanisms for promoting retention of the cell delivery article in tissue, the delivery assist mechanism(s) are held against a main portion of the cell delivery article to minimize an amount of drag that the delivery assist mechanism(s) assert against tissue during movement to a tissue site. In an embodiment, the delivery assist mechanism(s) are held in place mechanically and released after deployment at a tissue site. In an embodiment, a degradable coating covers the delivery assist mechanism(s), and the degradable coating is structured to release the delivery assist mechanism(s) after deployment at a tissue site.

In an embodiment, a standardized cell delivery article for use in multiple different treatment regimens includes at least one delivery assist mechanism which is incorporated for one or more of the treatment regimens, and the other treatment regimens are agnostic to whether or not the delivery assist mechanism is included. In this manner, manufacturing costs potentially may be reduced due to quantity of scale.

A time period for a delivery assist mechanism to retain a cell delivery article at a tissue site may range from about 24 hours to about 1 year, or more. For example, the delivery assist mechanism may be structured to retain the cell delivery article at the tissue site for about 24 hours, for about 2 days, for about 3 days, for about 4 days, for about 5 days, for about 6 days, for about one week, for about two weeks, for about three weeks, for about four weeks, for about five weeks, for about six weeks, for about seven weeks, for about 2 months, for about 3 months, for about 4 months, for about 5 months, for about 6 months, and so forth. In an embodiment, the delivery assist mechanism may be structured to retain the cell delivery article at the tissue site for one or more years.

### Cell Types

The cells contained within a cell delivery article described herein and delivered to various tissue sites may include various cell types. The cells may include a same cell type or different cell types. The cells may be autologous or allogeneic, or a combination of autologous and allogeneic. The cells being delivered may express cell products that are therapeutically beneficial agents. For example, hormones such as insulin, glucagon, parathyroid hormone, thyroid hormone, pituitary hormone, growth hormone, estrogen, progesterone, testosterone, or combinations thereof may be produced for use in a hormone therapy. Incretins such as gastric inhibitory peptide or glucagon-like peptide, or a combination of gastric inhibitory peptide and glucagon-like peptide may be produced. In an embodiment, the cells being delivered may produce factors such as cytokines and other factors involved with immune cell signaling. In some instances, the cells include cells that have been genetically modified to produce various substances. For example, stem cells may be genetically modified to produce hormones, growth factors, anti-tumor agents, or other active agents.

In an embodiment, the cells (instead of their expressed product) may be released from a cell delivery article to provide the therapeutic benefit. For example, the cells may include cells that have been genetically modified to recognize a specific molecule on a cancer cell (e.g., a CAR-T cell); these T-cells attack the cancer cell. In an embodiment, stem cells (modified or unmodified) are used to replace damaged or diseased tissue or organs.

In an embodiment, the cells included in a cell delivery article may include pancreatic islet cells, which may be alpha cells, beta cells, delta cells, genetically modified variants of any of the foregoing, or a combination thereof. The pancreatic islet cells may produce insulin, glucagon, somatostatin, or a combination thereof. Islets may be obtained from the subject's pancreas or a donor pancreas by separating the islets from exocrine fragments of the pancreas. Such separated islets may be procured from a supplier in bulk.

In an embodiment, the cells may include incretin cells, which may be K-cells, L-cell, I-cells, N-cells, S-cells, genetically modified variants of any of the foregoing, or a combination thereof. The incretin cells may produce gastric inhibitory peptide, glucagon-like peptide, or a combination thereof.

In an embodiment, the cells may include immune cells, which may be T-cells, B-cells, NK cells, macrophages, neutrophils, genetically modified variants of any of the foregoing, or a combination thereof. When an immune cell is a macrophage, the macrophage may produce TNF-alpha.

In an embodiment, the cells may include stem cells, such as embryonic stem cells, endothelial progenitor cells, hematopoietic stem cells, mesenchymal stem cells, neural stem cells, keratinocyte stem cells, other stem cells, genetically modified variants of any of the foregoing, or a combination thereof.

In an embodiment, the cells that may be delivered include chondrocytes, fibroblasts, or a combination thereof, such as to treat burn wounds or other open wounds, and conditions and diseases involving the joints.

The cells being delivered may be used, for example, in anti-cancer therapy (e.g., leukemia, lymphoma, multiple myeloma), used to replace damaged cells, used to produce factors or antibodies, or used to produce substances useful in healing tissue (e.g., to treat burns or to promote the growth of tissue to close large wounds).

In an embodiment, the cells being delivered produce one or more of: HB-EGF; FGFs 1, 2, and 4; PDGF; IGF-1; TGF-β1 and β2; TGF-β3; IL-1α and -β; IL-10; IL-4; IL-2, IL-12; IL-6, IL-8, IL-17a; LEP and LEPR; Endoglin; Adipoq; IGFBP1, IGFPB3; CSF1, CSF3 and receptor CSFR1; PPBP/NAP-2; HGF; NGRF; EGF; TNF-α.

In an embodiment, the cells being delivered produce one or more growth factors such as, but not limited to, one or more of: bFGF2 (basic fibroblast growth factor 2), bNGF (beta-nerve growth factor), FGF4 (fibroblast growth factor 4), FGF6 (fibroblast growth factor 6), FGF 9 (fibroblast growth factor 9), Fas ligand, IGFBP1 (insulin growth factor binding protein 1), IGFBP3 (insulin growth factor binding protein 3), IGFBP6 (insulin growth factor binding protein 6), LAP (transforming growth factor like), IGF-1 (insulin-like growth factor 1), IGF-2 (insulin-like growth factor 2), PDGF (platelet-derived growth factor), PDGFAA (platelet-derived growth factor Aα), PDGFAB (platelet-derived growth factor Aβ), PDGFBB (platelet-derived growth factor Bβ), TGFB1 (transforming growth factor β1), ANG (angiogenin), BDNF (brain-derived neurotrophic factor), BMP4 (bone morphogenic protein 4), BMP 6 (bone morphogenic protein 6), bNGF (beta-nerve growth factor), BTC (probetacellulin), CNTF (ciliary neurotrophic factor), EGF (epidermal growth factor), HGF (hepatocyte growth factor), hepatocyte-like growth factor, NT3 (neurotrophin 3), NT4 (neurotrophin 4), OPG (osteoprotegerin), Siglec5 (sialic acid binding If-like lectin 5), and TGF A (transforming growth factor alpha), TGF b1 (transforming growth factor beta 1), TGF b 2 (transforming growth factor beta 3), VEGF (vascular endothelial growth factor), VEGFD (vascular endothelial growth factor D), and PLGF (placental growth factor).

In an embodiment, the cells being delivered produce one or more chemokines such as, but not limited to, one or more of: CCL 2 (chemokine ligand 2), CCL 3 (chemokine ligand 3), CCL 4 (chemokine ligand 4), CCL 5 (chemokine ligand 5), CCL 7 (chemokine ligand 7), CCL 8 (chemokine ligand 8), CCL 13 (chemokine ligand 13), CCL 15 (chemokine ligand 16), CCL 17 (chemokine ligand 17), CCL 18 (chemokine ligand 18), CCL 19 (chemokine ligand 19), CCL 20 (chemokine ligand 20), CCL 22 (chemokine ligand 22), CCL 23 (chemokine ligand 23), CCL 24 (chemokine ligand 24), CCL25 (chemokine ligand 25), CCL 26 (chemokine ligand 26), CCL 27 (chemokine ligand 27), CCL 28 (chemokine ligand 28), CXC L1 (chemokine ligand 1), CXCL1/2/3 (chemokine ligand 1/2/3), CXCLS (CX chemokine ligand 5), CXCL9 (CX chemokine ligand 9), CXCL 10 (CX chemokine ligand 10), CXCL 13 (CX chemokine ligand 13), and CXCL 16 (CX chemokine ligand 16).

Given that a cell delivery article as described herein is a platform for cell delivery into a body, it is understood that the cell types included in the cell delivery article are not limited to those mentioned above, and that any desired cell type may be used.

By way of example, in an embodiment, a cell delivery article includes a reservoir containing pancreatic islet cells. The reservoir includes a reservoir outer wall having one or more pores. At least one plug that includes silicone and calcium peroxide is disposed within the cell delivery article in chemical communication with the reservoir. The calcium peroxide functions as an oxygen supply that is activated upon contact with water. A medium including an alginate gel is disposed within the reservoir and is adapted to support the pancreatic islet cells. The water used to activate the calcium peroxide may be provided by the alginate gel.

Any suitable number of cells may be included in a cell delivery article. The number of cells included may depend on factors such as a type of cell being delivered, a condition or disease being treated, a composition or dosage form in which the cell delivery article is formulated, and a route of administration. In general, hundreds to thousands of cells may be delivered to achieve a clinical effect.

In an embodiment, each cell delivery article may contain between about 100 cells and about 500 cells. For example, each cell delivery article may include about 100 cells, about 150 cells, about 200 cells, about 250 cells, about 300 cells, about 350 cells, about 400 cells, about 450 cells, or about 500 cells. In an embodiment, each cell delivery article may contain less than 100 cells. In an embodiment, each cell delivery article may include greater than 500 cells. In an embodiment, each cell delivery article may include greater than 1000 cells. In an embodiment, each cell delivery article may include greater than 10,000 cells. In an embodiment, each cell delivery article may include greater than 100,000 cells.

A dosage number of cells disposed in a cell delivery article may be selected according to a treatment plan. For example, in an embodiment of a treatment regimen, a cell delivery article containing islet cells is delivered to a subject body each day for a sequence of days (e.g., 90 days) or each week for a sequence of weeks (e.g., 30 weeks) by a selected composition and dosage form. The subject's health indicators (e.g., blood glucose or other indicator) can be monitored during the treatment regimen and/or after the treatment regimen to identify whether additional cell delivery may be beneficial to reach glycemic control. It has been estimated that for successful islet infusion into the liver to replace pancreatic function, at least 5,000 islets per kilogram of body weight is needed to be delivered (e.g., at least 300,000 islets); it is expected, without being bound by theory, that a number of islets to be delivered into the peritoneal cavity to replace pancreatic function would be a similar number, and fewer may be needed if the cells are augmenting rather than replacing pancreatic function. It is further expected that fewer cells may be needed if the cells have a high viability percentage as obtained from a supplier, in comparison to certain supplies of cells which have 30%-40% viability.

In this disclosure, articles and corresponding methods are presented in which hundreds or thousands of cells may be delivered in a single dose. The dose may be delivered by any suitable route.

By providing cells which can be maintained within a body to express cell products, treatment may be personalized to individual needs, such as with a goal to restore natural function to the body.

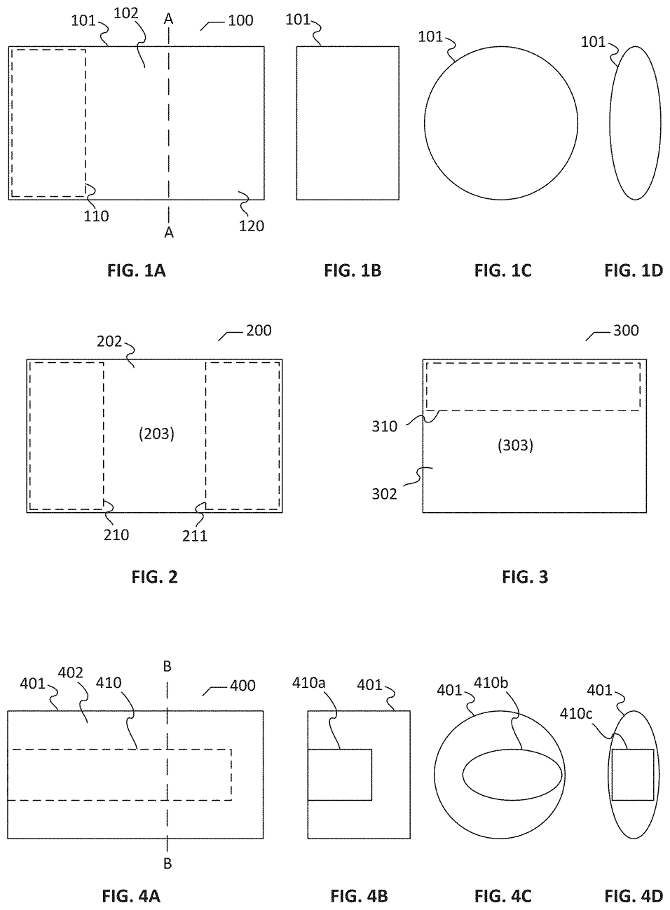

Turning now to the figures, FIG. 1 through FIG. 13 illustrate a few of the embodiments of the present disclosure. Other embodiments will be apparent by reviewing the text and figures of the present disclosure.

FIG. 1A illustrates in side view an embodiment of a cell delivery article 100 for delivering cells according to the present disclosure. Article 100 has an outer perimeter 101, and defines a cavity 102. Article 100 includes a plug 110 disposed within cavity 102. Article 100 further includes a reservoir 120 disposed in cavity 102, or reservoir 120 is a portion of cavity 102 as limited by outer perimeter 101 and plug 110. FIG. 1B, FIG. 1C, and FIG. 1D illustrate various cross-sectional shapes that may be defined by article 100 at cut line A-A; other cross-sectional shapes are also encompassed by the present disclosure, and cross-sectional shape may vary along a length (e.g., along an axis perpendicular to cut line A-A) of article 100. FIG. 1B illustrates a rectangular cross-sectional shape; FIG. 1C illustrates a circular cross-sectional shape; and FIG. 1D illustrates an elliptical cross-sectional shape. More generally, a cross-sectional shape at a position along a length of article 100 may have one or more sides, the sides may be of similar lengths or different lengths, and each side may have a straight, arcuate, or irregular shape.

In an embodiment, portions of article 100 (or other cell delivery articles described in the present disclosure) are formed of PLA or PGA, or a combination of PLA and PGA (e.g., PLGA or PGLA). In an embodiment, portions of article 100 are formed of another polymer or a combination of polymers, where such polymers may be naturally-occurring or synthetic. Selection of materials to use for article 100 may be based in part on a desired degradation profile for article 100 for a particular treatment. For example, materials may be selected for degradation of article 100 in a matter of hours or days, or for degradation of article 100 in a matter of weeks, months, or years.

FIG. 2 illustrates in side view an embodiment of a cell delivery article 200 defining a cavity 202 in which two plugs 210, 211 are disposed. Article 200 with plugs 210, 211 defines a void 203 within cavity 202 in which a reservoir may be disposed, or void 203 is a reservoir within which cells may be disposed.

FIG. 3 illustrates in side view an embodiment of a cell delivery article 300 defining a cavity 302 in which a plug 310 is disposed. Article 300 with plug 310 defines a void 303 within cavity 302 in which a reservoir may be disposed, or void 303 is a reservoir within which cells may be disposed.

FIG. 1, FIG. 2, and FIG. 3 illustrate plugs (plugs 110, 210, 211, 310) that are positioned adjacent to an outer edge of the respective cell delivery article. In other embodiments, a plug may be disposed at any location within a cell delivery article.

FIG. 4A illustrates in side view an embodiment of a cell delivery article 400 for delivering cells according to the present disclosure. Article 400 has an outer perimeter 401, and defines a cavity 402. Article 400 includes a plug 410 disposed within cavity 402. In this embodiment, a reservoir may be disposed in cavity 402 adjacent to or surrounding plug 410, or a reservoir is a portion of cavity 402 as limited by edges of article 400 and plug 410. Plug 410 may be floating within the reservoir, or may be affixed at one end or both ends or along an edge. FIG. 4B, FIG. 4C, and FIG. 4D illustrate various cross-sectional shapes that may be defined by article 400 and plug 410 at cut line B-B; other shapes are also encompassed by the present disclosure, and cross-sectional shape may vary along a length (e.g., along an axis perpendicular to cut line B-B) of article 400. FIG. 4B illustrates a rectangular cross-sectional shape of article 400 with a rectangular cross-sectional shape of plug 410 (shown as plug 410a); FIG. 4C illustrates a circular cross-sectional shape of article 400 with an elliptical cross-sectional shape of plug 410 (shown as plug 410b); and FIG. 4D illustrates an elliptical cross-sectional shape of article 400 with a rectangular cross-sectional shape of plug 410 (shown as plug 410c). More generally, a cross-sectional shape of either article 400 or plug 410 at a position along a length of article 400 may have one or more sides, the sides may be of similar lengths or different lengths, and each side may have a straight, arcuate, or irregular shape.

Although embodiments of cell delivery articles as illustrated in FIG. 1A, FIG. 2, FIG. 3 and FIG. 4A have an outline that is a parallelogram for convenience, other shapes are with the scope of the present disclosure.

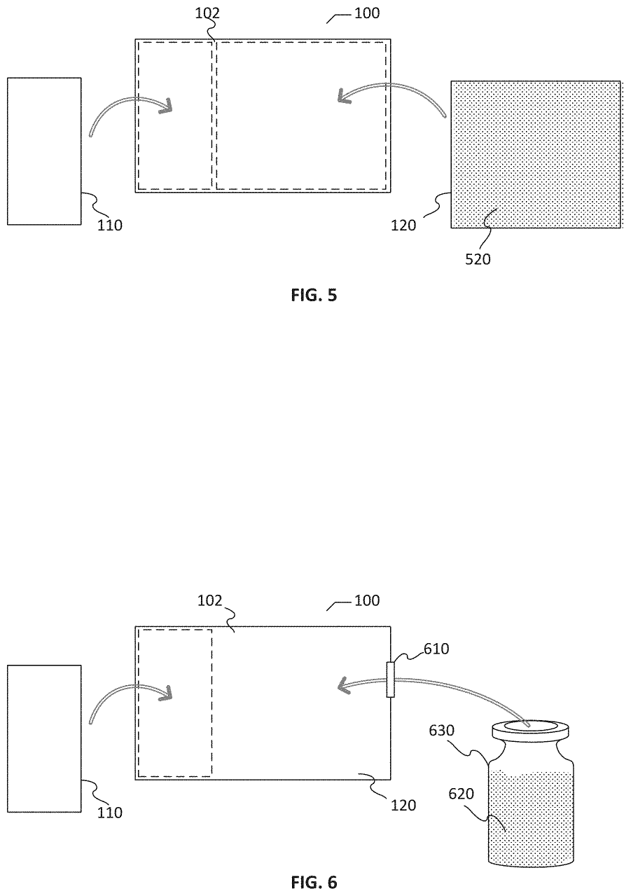

FIG. 5 illustrates in side view an embodiment of cell delivery article 100 as shown in FIG. 1A, prior to assembly of article 100. In this embodiment, plug 110 and reservoir 120 may be at least partially manufactured separately from a remainder of article 100, and then disposed in article 100. For example with respect to plug 110 when formed of silicone: a mass of silicone may be formed into a desired shape to form plug 110, then plug 110 is disposed in cavity 102 and infused with an oxygen supply component; or a mass of silicone may be formed into a desired shape to form plug 110 and infused with an oxygen supply component, then plug 110 is disposed in cavity 102; or a mass of silicone is disposed in cavity 102 and pressed into a desired shape against an interior perimeter of article 100, then infused with an oxygen supply component; or a mass of silicone is infused with an oxygen supply component, then is disposed in cavity 102 and pressed into a desired shape against an interior perimeter of article 100. For example with respect to reservoir 120, a suitable container may be obtained or formed, a supply of cells 520 may be disposed into the container with a medium (the cells being disposed before, concurrently with, or after the medium), and reservoir 120 is positioned in cavity 102 of article 100.

FIG. 6 illustrates in side view an embodiment of cell delivery article 100 as shown in FIG. 1A, prior to assembly of article 100. In this embodiment, plug 110 may be at least partially manufactured separately from a remainder of article 100 and then disposed in article 100, such as described with respect to FIG. 5. Article 100 with plug 110 defines reservoir 120 in this embodiment, and article 100 includes a port 610 providing access to reservoir 120. Here, cells 620 are provided in a container 630 (e.g., vial or other container of a size suitable for the manufacturing technique applied), and are placed into cavity 120 of article 100 through port 610, such as with a pipette or syringe, or using a funnel, or other technique. A medium may be dispensed into cavity 120 before, concurrently with, or after cells 620 are placed into cavity 120. In an embodiment, cells 620 are suspended in a medium in container 630 prior to being placed into cavity 120. Mediums and cell types are described elsewhere herein.

As discussed above, a cell delivery article according to the present disclosure may optionally include a delivery assist mechanism structured to promote movement of the cell delivery article into and/or through tissue, and/or to aid in retention of the cell delivery article at an intended tissue site.

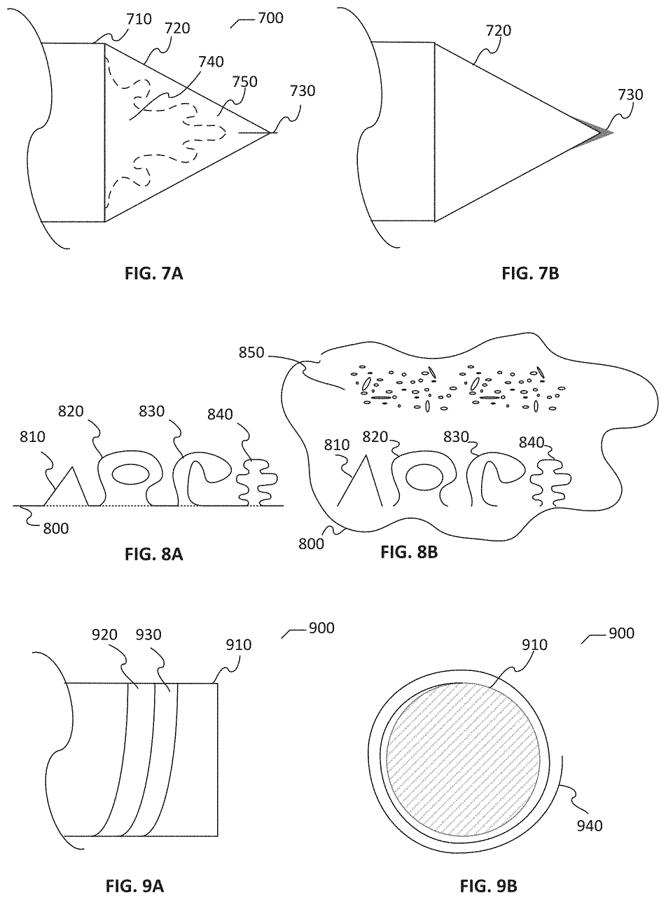

FIG. 7A illustrates in side view a cell delivery article 700 having a main portion 710 (e.g., similar to articles 100, 200, 300, 400 illustrated and described with respect to FIG. 1A, FIG. 2, FIG. 3, FIG. 4A, FIG. 5, or FIG. 6) and a delivery assist mechanism 720 including a tip 730. In an embodiment, delivery assist mechanism 720 is formed together with article 700. In an embodiment, delivery assist mechanism 720 is formed separately from article 700 and is attached to article 700, such as by applying heat or a vibration force to meld article 700 and delivery assist mechanism 720, or such as by applying an adhesive substance or a double-sided adhesive tape between article 700 and delivery assist mechanism 720. In an embodiment, delivery assist mechanism 720 is solid throughout; in another embodiment, delivery assist mechanism 720 defines a cavity. In an embodiment, delivery assist mechanism 720 is formed from multiple materials. In one such embodiment, a core 740 is covered by a coating 750, where coating 750 degrades after deployment of article 700, leaving core 740 exposed. Core 740 is shaped in a manner to initially anchor in tissue and subsequently promote tissue growth around core 740, thus providing short-term retention and long-term retention of article 700 in tissue. In this embodiment, delivery assist mechanism 720 first promotes movement of the cell delivery article into and/or through tissue by way of the angled shape of delivery assist mechanism 720 in concert with tip 730, and then aids in retention of the cell delivery article at a tissue site by way of an anti-movement shape initially, and subsequently tissue growth around core 740 (after coating 750 degrades).

FIG. 7B illustrates an embodiment of article 700 of FIG. 7A after a 90-degree rotation along a lengthwise axis of article 700. In this embodiment, tip 730 protrudes from or beyond delivery assist mechanism 720, to provide a sharp interface between article 700 and tissue at a delivery site, to assist penetration of article 700 into and through tissue at the tissue site.

FIG. 8A illustrates examples of embodiments of delivery assist mechanisms which are formed on, or attached to, a cell delivery article 800, as seen from a side view when the delivery assist mechanisms are allowed to deploy away from article 800 (while each delivery assist mechanism remains attached to article 800 along a section of the delivery assist mechanism). Delivery assist mechanism 810 is triangularly-shaped, which may resist movement in a direction perpendicular its face, such as movement within a range of angles between −60 degrees to +60 degrees from the perpendicular, as well as providing a surface for tissue growth to occur for long-term retention. Delivery assist mechanism 820 is a flap defining a hole, where the flap can resist movement such as described with respect to delivery assist mechanism 810, and tissue growth can occur around delivery assist mechanism 820 and through its hole. Delivery assist mechanism 830 is a flap having a hooked shape, where the flap can resist movement such as described with respect to delivery assist mechanism 810, and tissue growth can occur around the hooked shape. Delivery assist mechanism 840 is a flap having an irregular shape, where the flap can resist movement such as described with respect to delivery assist mechanism 810, and tissue growth can occur around the irregular shape.

FIG. 8B illustrates an example of how the delivery assist mechanisms 810, 820, 830, 840 of FIG. 8A might be cut into a material at a surface of article 800. The delivery assist mechanisms may be retained in position until article 800 is delivered to a tissue site, and then allowed to deploy away from the surface (as illustrated in FIG. 8A) to retain article 800 in the tissue. In an embodiment, delivery assist mechanism 810 or similar may be cut at an angle into the material at the surface of article 800 (or other cell delivery article) to achieve a protrusion resembling a fish scale. Also illustrated in FIG. 8B are holes and slits (in an area indicated as area 850) formed in a material at the surface of article 800 before or after positioning the material onto article 800. Such holes and slits may promote tissue growth onto the surface without protrusions away from the surface. A few examples of delivery assist mechanisms to promote tissue growth are illustrated and described with respect to FIG. 8A and FIG. 8B; many other shapes and sizes of delivery assist mechanisms are within the scope of the present disclosure, and various shapes and sizes may be used on a single cell delivery article.

FIG. 9A illustrates an embodiment of a cell delivery article 900 having a portion 910 around which delivery assist mechanisms 920, 930 are wrapped, or are cut into a material at a surface of the portion 910. Until article 900 is positioned at a tissue site, delivery assist mechanisms 920, 930 are held against portion 910 (e.g., by a coating that degrades when in contact with tissue) and are allowed to deploy circumferentially outwards after positioning at the tissue site. In an embodiment, delivery assist mechanisms 920, 930 are a single delivery assist mechanism 940 as illustrated in FIG. 9B, which deploys to form a continuous piece helically wrapped around a length of portion 910 of article 900.

Delivery assist mechanisms can be formed of any suitable material, such as, for example, one or more naturally-occurring or synthetic polymers. In an embodiment, a delivery assist mechanism is formed of polyethylene oxide (PEO). A tip (e.g., tip 730) can be formed of a hard material which can retain a sharp point, such as a metal, a ceramic, or other material or a combination of materials. In an embodiment, a tip is formed of magnesium.

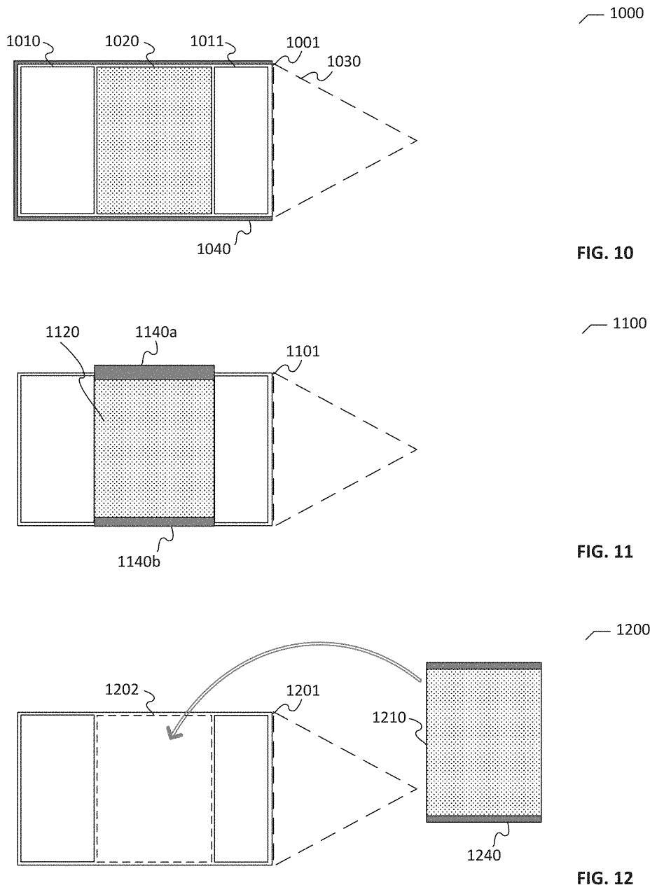

FIG. 10 illustrates in cross-section an embodiment of a cell delivery article 1000 having a main portion 1001 and including two plugs 1010, 1011 and a reservoir 1020 disposed in a cavity defined by main portion 1001. Article 1000 optionally includes a conical delivery assist mechanism 1030 integrated with or attached to main portion 1001. A bio-ghost coating 1040 covers an exposed portion of main portion 1001. In embodiments including optional delivery assist mechanism 1030, bio-ghost coating 1040 may additionally cover delivery assist mechanism 1030; in an embodiment, delivery assist mechanism 1030 is left uncovered to provide for retention of article 1000 in tissue by growth of tissue around delivery assist mechanism 1030, or to promote degradation of delivery assist mechanism 1030.

FIG. 11 illustrates in cross-section an embodiment of a cell delivery article 1100 similar to cell delivery article 1000 in FIG. 10, except that a bio-ghost coating 1140 (labeled 1140a and 1140b) covers an outer wall of a reservoir 1120, leaving a remainder of a main portion 1101 of cell delivery article 1100 exposed to tissue. In this manner, cells within reservoir 1120 are protected from attack by immune cells in the tissue, fibrotic development can be minimized or prevented on reservoir 1120, and article 1100 provides surface area on which tissue growth can occur to aid in retention of article 1100 in tissue. As also illustrated in FIG. 11, bio-ghost coating 1140 may protrude from main portion 1101, such as bio-ghost coating 1140a, or may be substantially colinear with a surface of main portion 1101, such as bio-ghost coating 1140b, or may be recessed from a surface of main portion 1101 (not shown).

FIG. 12 illustrates in cross-section an embodiment of a cell delivery article 1200 similar to cell delivery article 1100 in FIG. 11, except that a bio-ghost coating 1240 is applied to a reservoir 1210 before reservoir 1210 is disposed in a cavity 1202 defined by a main portion 1201 of article 1200. Although shown as covering a portion of an outer perimeter of reservoir 1210 which will be exposed outside of article 1200, bio-ghost coating 1240 may cover additional surfaces, or all of, reservoir 1210.

In any of the embodiments in FIG. 10, FIG. 11, or FIG. 12, or any other embodiments of the present disclosure, a cell delivery article may include a degradable coating over all of, or over a portion of, the cell delivery article (not shown in the illustrations), to delay action of the cell delivery article for a designed time.

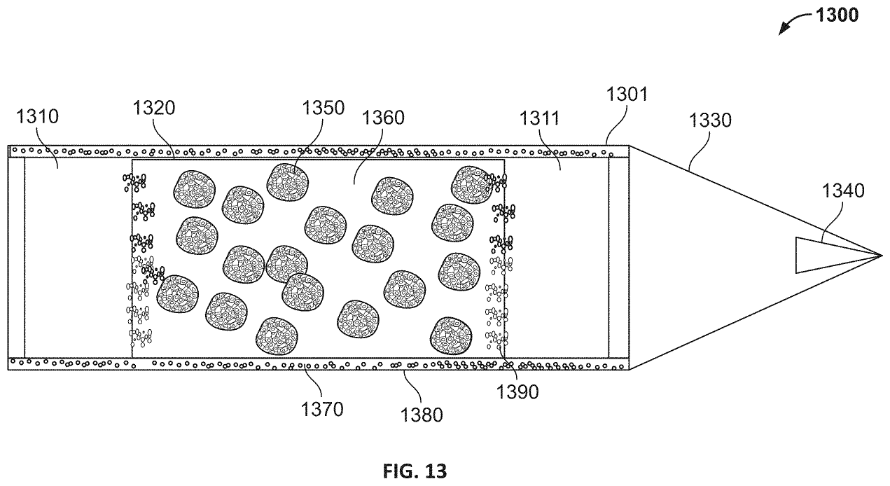

FIG. 13 illustrates in cross-section a design of an embodiment of a cell delivery article 1300 according to the present disclosure. Cell delivery article 1300 has a main portion 1301 that defines a cavity in which two plugs 1310, 1311 are disposed (e.g., formed in or added to the cavity) and a reservoir 1320 is disposed (e.g., added to the cavity or defined by the cavity and plugs 1310, 1311). In an embodiment, plugs 1310, 1311 are formed of, or include, silicone, and an oxygen supply component (e.g., calcium peroxide) is disposed within the silicone. Article 1300 further includes a delivery assist mechanism 1330 incorporated with or attached to main portion 1301. In this embodiment, delivery assist mechanism 1330 includes a sharp tip 1340. In an embodiment, delivery assist mechanism 1330 is formed of PEO, and tip 1340 is formed of magnesium. Cells 1350 are disposed in reservoir 1320 with a medium 1360. In an embodiment, medium 1360 is an alginate gel. In an embodiment, the cells include islet cells. A porous outer wall extends at least across reservoir 1320 and forms reservoir outer wall 1370 in which the pores are sized to block immune system cells (e.g., lymphocytes, neutrophils, monocytes, macrophages, etc.) and proteins (e.g., cytokines, antibodies, etc.) from entering reservoir 1320, while allowing water and nutrients to enter reservoir 1320, allowing interstitial fluid (e.g., containing insulin, glucose, etc.) to enter reservoir 1320, and allowing cell products to exit reservoir 1320. A coating 1380 covers at least reservoir outer wall 1370 and may cover other portions of main portion 1301 of article 1300. In an embodiment, coating 1380 is a bio-ghost coating to avoid an immunosuppressive response and fibrotic development. In an embodiment, coating 1380 includes a biomimetic synthetic peptide (e.g., a multi-arm peptide, or MAP) that is an analogue of the cell binding domain of collagen. The biomimetic synthetic peptide is covalently attached to portions of article 1300. In an embodiment, coating 1380 includes a degradable coating layer. Until the oxygen supply component is depleted, water molecules entering plugs 1310, 1311 from medium 1360 (e.g., either originally from medium 1360 or received into reservoir 1320 through reservoir outer wall 1370 from tissue) combine with the oxygen supply component and release oxygen 1390, which is provided back to medium 1360 and reaches cells 1350 to sustain cells 1350. In this way, article 1300 can maintain viability of cells 1350 for a time, such as until cells 1350 are incorporated into a tissue site.

In an embodiment, article 1300 may be stored at low temperature to slow release of oxygen from oxygen supply components in plugs 1310, 1311 until article 1300 is released into a subject body.

In an embodiment, calcium peroxide powder is mixed with silicone and extruded to form plugs 1310, 1311; extrusion may be into a shape mold for transfer into article 1300, or extrusion may be directly into article 1300.

In an embodiment, reservoir outer wall 1370 is a membrane. The membrane may be, or may include, for example, ePTFE, a porous polyimide, polysulfone, cellulose, or a combination of two or more of the foregoing. In an embodiment, the membrane is porous polyimide that is plasma treated to functionalize the surface for bonding a MAP onto the membrane.

By way of example with respect to bio-ghost coatings, FIG. 14 illustrates a stretch of a collagen fiber with examples of P-15 peptide binding domains of the collagen indicated. These P-15 cell binding bumps form rings on collagen fibers approximately every 74 nanometers. The P-15 peptides can be synthesized to formulate a bio-ghost coating of a P-15 analogue.

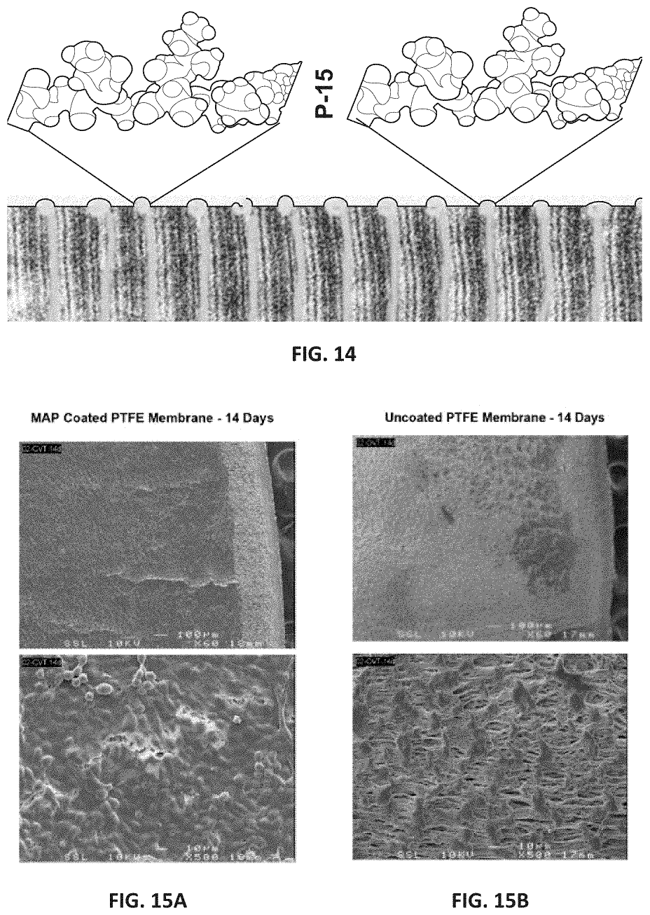

An example of an efficacy of a bio-ghost coating in attracting body cells to bind to the bio-ghost coating is illustrated in SEM images in FIG. 15A, showing cells migrated from a culture vessel onto a luminal surface of an ePTFE, capillary placed vertically in the culture vessel, as compared to a lack of cell migration onto an uncoated membrane shown in the SEM images in FIG. 15B.

### ARTICLE COMPOSITIONS AND DOSAGE FORMS

The cell delivery articles described herein may be formulated into any suitable composition and the composition provided in any suitable dosage form. Suitable carriers and/or excipients may be employed for the desired composition and dosage form. A composition may include one or more cell delivery articles, and may be tailored to a particular indication or use. For example, a composition may be tailored for oral delivery, topical delivery, delivery by injection, or intravenous delivery.

Oral delivery dosage forms include, but are not limited to, liquids, suspensions, capsules, tablets, and dissolvable films.

Topical delivery dosage forms include, but are not limited to, gels, pastes, ointments, creams, serums, lotions, emulsions, sprays, solutions, aerosols, films, patches, bandages, eye drops, ear drops, and spreadable film-forming compositions.

Injectable delivery dosage forms include, but are not limited to, liquids or suspensions that may be delivered via a syringe, image-guided needle, or other needled tool.

Intravenous delivery dosage forms include, but are not limited to, liquids or suspensions.

A composition may be one or more cell delivery articles without additional components (e.g., without carriers or excipients). Such a composition may be delivered by any suitable dosage form.

A composition and/or dosage form may be formulated for immediate, sustained, or controlled release of the cell delivery articles, the cells contained therein, or the expressed cell products. A release time may be designed, such as by adjusting a degradation rate of the dosage form or the cell delivery article, either via manipulation of the materials used to make them, by altering material thicknesses, by including coatings on the cell delivery article or dosage form, or other delayed release mechanism.

In an embodiment, a dosage form includes a transporter, which is positioned in a subject body and is then activated to expose the composition to tissue; in this way, one or more cell delivery articles in the composition can be situated such that cells in the cell delivery articles can be incorporated into the tissue site. The transporter may be self-activated to expose the composition to tissue.

In an embodiment of a self-activated transporter for oral delivery, a capsule contains a balloon; when the capsule is exposed to tissue with a pH value in a particular range (e.g., above 5.5, below 7.0, between 5 and 7, etc.), an outer portion of the capsule begins to degrade, biological matter (or digestive matter) eventually breaches the outer portion of the capsule and reaches the balloon, the balloon responds to the biological matter (or digestive matter) by inflating, and the inflation triggers a mechanism that expels the composition out of the transporter. In an embodiment of such a self-activated transporter, the capsule begins to degrade at a pH value present in the small intestine, and the composition is expelled into a wall of the small intestine; for example, depending on the expulsion force applied, the composition may be expelled into the mucosa, submucosa, muscularis, serosa, or other layer of the intestinal wall, or into the peritoneum or the peritoneal cavity or into an organ in the peritoneal cavity. For example, the composition can include a cell delivery article carrying islet cells in a reservoir of the cell delivery article, and the cell delivery article can include a delivery assist mechanism to aid in penetration of the cell delivery article into the intestinal wall, or through the intestinal wall into the peritoneal cavity; when the islet cells are incorporated into the tissue site, they can provide pancreatic-like functionality to the subject body.

In an embodiment, a transporter expels a cell delivery article with sufficient force to deliver the cell delivery article into (or through) a wall of the GI tract whether the subject is in a fasted state or a non-fasted state; in other words, the transporter is structured to deliver the cell delivery article through digestive matter if present in the GI tract.

In an embodiment, a transporter may incorporate electronics, such as to detect a position or movement trajectory of the transporter, to detect when a cell delivery article is exposed to a tissue site, to detect when a transporter has actuated to expel a cell delivery article, to detect environmental conditions (e.g., temperature, pressure, humidity, or pH level), to communicate with a device external to the transporter (including external to the subject body), or other function. The electronics may include a memory device to store information. Information stored in the memory device may be relayed to a device external to the transporter by way of a communication interface included in the electronics.

In an embodiment, a cell delivery article may incorporate electronics, such as to detect a position or movement trajectory of the cell delivery article, to detect when the cell delivery article is exposed to a tissue site, to detect when the cell delivery article is expelled from a transporter, to detect environmental conditions (e.g., temperature, pressure, humidity, or pH level), to communicate with a device external to the cell delivery article (including external to the subject body), or other function. The electronics may include a memory device to store information. Information stored in the memory device may be relayed to a device external to the cell delivery article by way of a communication interface included in the electronics.

### METHODS

Some of the methods described herein relate to delivering cells to tissue sites using a cell delivery article capable of maintaining cell viability for a time period that allows the cells to express cell products and incorporate into a tissue site. In an embodiment, such an article is further capable of hiding from the immune system through bio-ghosting. Other methods may include administering the cells to a subject according to a dosing schedule. The cells may be administered by any suitable route, for example, oral or parenteral routes. Dosage forms including one or more cell delivery articles may generally be formulated based on an intended route of delivery or a desired dosing schedule.

### Methods of Delivery

Introduction of a cell delivery article into a subject may be accomplished in various ways. For example, the cell delivery article may be introduced orally, rectally, intravenously, intramuscularly, subcutaneously, intradermally, intraperitoneally, intrathecally, intra-articularly, intraocularly, or topically to a tissue site. Injection is generally used to introduce the cell delivery articles intravenously, intramuscularly, subcutaneously, intradermally, intraperitoneally, intrathecally, intra-articularly, and intraocularly. However, topical application may also be employed to introduce the cell delivery article into the eye, subcutaneous tissue, or dermal tissue. In an embodiment, the cell delivery articles are introduced into a subject via direct injection near or adjacent to pathology of interest, for example, a cancer or an infected area.

The cell delivery articles described herein may be delivered by any suitable route. Oral routes typically include delivery by mouth. Here the cell delivery article may be contained within a transporter, which would enter the GI tract and release the article within the GI tract. The article may penetrate or become attached to a tissue site in the intestine, such as with the aid of a delivery assist mechanism. During transit in the intestine, the cells within the cell delivery article are generally kept viable by the nutrient-containing medium in which they are dispersed, as well as a supply of oxygen furnished by a chemical reaction between the medium and an oxygen supply component in a plug of the cell delivery article. An oral route of delivery for the cell delivery articles may be selected, for example, when the intended tissue site is within the GI tract or reachable by delivery within the GI tract (e.g., through a wall of the GI tract into the peritoneum), and a convenient mode of administration is desired.

In an embodiment, an oral route of delivery is used to deliver cells into a wall of the GI tract (e.g., a wall of the esophagus, stomach, small intestine, large intestine, colon, etc.); in an embodiment, a force used by a transporter for delivery into the wall of the GI tract results in delivery into a wall of the GI tract, the peritoneum, or the peritoneal cavity or an organ therein.

Parenteral routes of delivery generally include all non-oral routes. Parenteral routes may include, for example, intramuscular administration, topical administration, subcutaneous administration, intradermal administration, rectal administration, intravenous administration, intraperitoneal administration, intrathecal administration, intra-articular administration, and intraocular administration. The parenteral route of delivery may be selected, for example, when the subject cannot tolerate oral delivery, when hepatic first-pass metabolism is to be avoided, and/or when local delivery of the cell delivery article is desired. Parenteral routes of delivery may include the use of a transporter containing a cell delivery article.

Methods for delivering cells to tissue sites may include introducing a cell delivery article into a body of a subject, where the cell delivery article includes cells within a reservoir. The reservoir may include a reservoir outer wall having one or more pores. A medium disposed within the reservoir is adapted to support the cells. The cell delivery article includes an oxygen supply component in one or more plugs disposed in the cell delivery article. A bio-ghost coating covering the reservoir outer wall may be provided, which may prevent recognition of the cell delivery article by the subject's immune system and minimize or prevent fibrotic development. The bio-ghost coating may include a biomimetic peptide; for example, the multi-arm peptide P-15. This multi-arm peptide may be an analogue of the cell binding domain of collagen.

The method may also include generating oxygen from an oxygen supply component to assist in the support of the cells, and retaining the cell delivery article at the tissue site using a delivery assist mechanism at least until the cells are incorporated into the tissue site and are capable of expressing their cell product.

The cell delivery articles may be delivered to any tissue site. For example, the tissue site may be the small intestine, the large intestine, the colon, the liver, an intraportal vein, a kidney capsule, the omentum, the peritoneum, the peritoneal cavity, an ovary, the uterus, the thyroid, the brain, the intrathecal space, skin, muscle, an epididymal fat pad, subcutaneous tissue, a blood vessel, an arteriovenous site, an eye, or other tissue site.

The methods may further include maintaining the cell delivery article at the tissue site for a time period sufficient to allow incorporation of the cells into the tissue site. Maintaining the cell delivery article may include controlling a degradation rate of the cell delivery article. The time period in which the cell delivery article is maintained at the tissue site may range from about one or two days to about six months, or longer.

In some instances, maintaining includes retaining the cell delivery article at the tissue site using a delivery assist mechanism.

Cells that may be delivered with a cell delivery article may include various cell types. In an embodiment, the cells include a same cell type. In an embodiment, the cells include different cell types. The cells may be autologous or allogeneic, or a combination of the foregoing. Cell types are discussed in detail elsewhere in the present disclosure.

### Methods of Administration

The cell delivery articles described herein may be administered according to any suitable dosing schedule. Dosing may consider factors such as route of delivery, type of cell being delivered and/or particular cell product being made, severity of a condition or disease being treated, whether the dosing schedule is for providing maintenance levels of a cell product or for providing a loading dose, and/or subject compliance.

In general, methods include providing a treatment regimen for a condition, the treatment regimen including a dosing schedule, and administering a dose, the dose including one or more cell delivery articles in a dosage form according to the dosing schedule. The one or more cell delivery articles typically include cells, a medium, and an oxygen supply component for supporting the cells.

Dosing of the cell delivery articles may be influenced by several general principles associated with the various routes of delivery. However, it is often difficult to predict the clinical effect a substance may have in a subject, requiring that the dosing regimen be tailored for each subject. Accordingly, the dosing of the cell delivery articles described herein may implemented in any suitable fashion, and may be adjusted based on the clinical effect observed (e.g., a blood concentration of the cell product, reduction in symptoms, lack of clinical response, etc.)

Some variations of the method include a dosing schedule that administers the dose periodically. In other variations, the dosing schedule includes administering the dose once a day or multiple times per day for a predetermined number of days. In further variations, the dosing schedule includes administering the dose once a week for a predetermined number of weeks. In another variation, the dosing schedule includes administering the dose once a month for a predetermined number of months. The dosing schedule may be continued until the desired number of cells are delivered and/or until a clinical effect is achieved. In an embodiment, a loading dose may be delivered, followed by maintenance doses.

A dose including one or more cell delivery articles may be administered in various ways. For example, and as previously described, the dose may be administered orally, rectally, intravenously, intramuscularly, subcutaneously, intradermally, intraperitoneally, intrathecally, intra-articularly, intraocularly, or topically. In an embodiment, the doses are administered to the subject via direct injection near or adjacent to pathology of interest, for example, a cancer or an infected area.

The dose may be formulated into any suitable dosage form. The dosage form may be tailored to the particular indication of use. For example, the dosage form may be tailored for oral delivery and include pancreatic islet cells when used to treat diabetes mellitus or pancreatitis. In addition to oral dosage forms, the cell delivery articles may be formulated as a topical composition, an injectable composition, or an intravenous composition. Suitable carriers and/or excipients may be employed based on the dosage form being made. One or more cell delivery articles may be included in a dosage form. A dosage form may be structured for immediate, sustained, or controlled release of one more cell delivery articles contained in the dosage form.

Various conditions or disorders may be treated with the cell delivery articles described herein. Such conditions or disorders include without limitation, diabetes, pancreatitis, cancer, thyroid disease, growth deficiency, and neurological disease. In some instances, the condition or disorder is a burn. In other instances, the condition is a wound or skin defect.

When the cells includes pancreatic islet cells, the treatment regimen may further include evaluating one or more indicators of pancreatic health of the subject, and either sustaining the treatment regimen if the evaluation does not indicate pancreatic health, or revising the treatment regimen if the evaluation does indicate pancreatic health. When a different cell type is administered, the treatment regimen may still include evaluating one or more indicators relating to the clinical effect of the cell or cell product to determine whether the treatment regimen should be adjusted.

In an embodiment of a treatment regimen, a cell delivery article (e.g., one of the cell delivery articles illustrated in FIG. 1A-FIG. 13) is delivered to a tissue site of the subject each day (or at other periodic or non-periodic interval). The subject is occasionally (e.g., daily, weekly, monthly) tested for one or more indicators of pancreatic health (e.g., pancreatic enzymes, fat level, inflammation, glucagon level, reaction to glucose clamp, etc.). When the medical practitioner determines that the implanted pancreatic islets are functioning sufficiently, dosing of islets is discontinued. A maintenance dose or repeat treatments may be used when needed.

In an embodiment of a cell delivery article and corresponding treatment regimen, the cell delivery article (e.g., one of the cell delivery articles illustrated in FIG. 1A-FIG. 13) is made to include 300-500 islet cells. The cell delivery article is incorporated into an oral dosage form (e.g., a capsule) capable of expelling the cell delivery article into tissue of the GI tract, such that the cell delivery article is implanted in a subject body. The expulsion force may be designed to expel the cell delivery article into a wall of the GI tract, or to expel the cell delivery article through the wall of the GI tract and into the peritoneum or the peritoneal cavity. The oral dosage form is ingested by a subject to deliver a dose of islet cells by expulsion of the cell delivery article from the oral dosage form. The subject subsequently ingests one oral dosage form each day for several months. Occasionally, or periodically (e.g., once per month), the subject is tested, and dosing is discontinued after sufficient pancreatic function is demonstrated.

Although described with respect to pancreatic islet cell delivery, a cell delivery article is suitable for delivering other cell types, additionally or alternatively to islet cells, in accordance with a treatment plan.

In an embodiment, a treatment plan includes delivering one or more cell delivery article containing one more cell types to a body of a subject once per week for an initial period, and based on a response of the body of the subject, either continuing with the once per week delivery, discontinuing the once per week delivery, reducing a dosage amount per cell delivery article, reducing a number of cell delivery articles delivered each week, or modifying the treatment plan to extend the delivery period from one week, such as extending to twice monthly, monthly, every two months, quarterly, bi-annually, or annually. In other embodiments, an initial delivery period may be daily, monthly, every two months, quarterly, or other period.

In an embodiment, delivery of a cell delivery article is orally. In an embodiment, delivery of a cell delivery article is intravenous. In an embodiment, delivery of a cell delivery article is intraperitoneal. In an embodiment, delivery of a cell delivery article is cutaneous or subcutaneous.

In an embodiment, a cell delivery article contains incretin cells. In an embodiment, a cell delivery article contains immune cells. In an embodiment, a cell delivery article contains stem cells.

### EXAMPLES

The following examples are illustrative only and should not be construed as limiting the disclosure in any way.

### Example 1

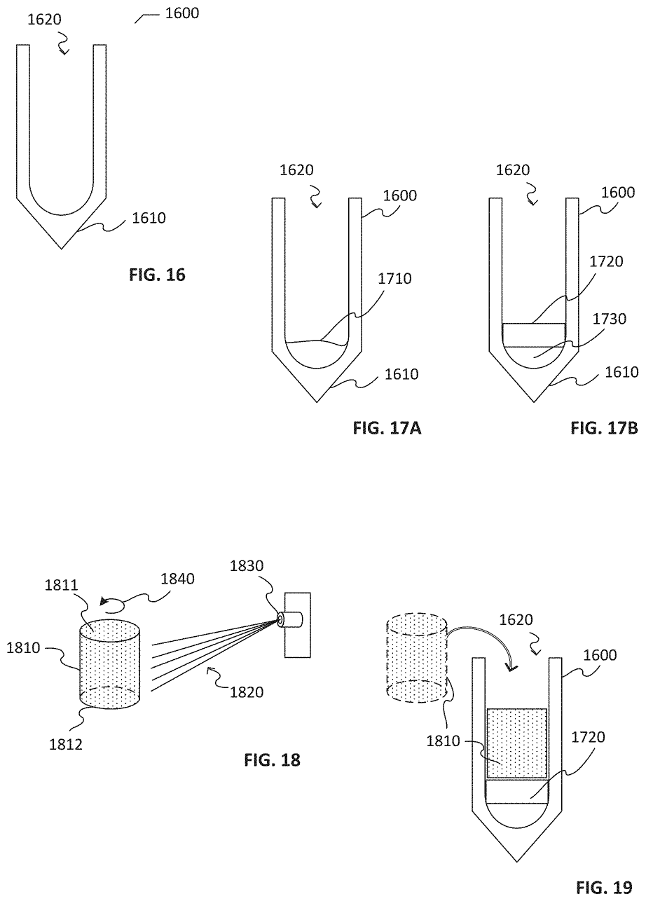

FIG. 16-FIG. 24B illustrate embodiments of methods for manufacturing a cell delivery article in accordance with the present disclosure.

FIG. 16 illustrates, in cross-section along a lengthwise axis, an embodiment of a shell 1600 which is preformed (e.g., formed by injection molding) and provided in singular form or in an interconnected row or matrix of such shells. Shell 1600 in this embodiment has a rigid or semi-rigid framing portion such that shell 1600 generally substantially retains its shape after formation. In this embodiment, shell 1600 is formed from PGA, PLA, PLGA, or PGLA, and other materials may also be included. A selection between PGA, PLA, PLGA, and PGLA depends in part on manufacturability and in part on how long it is desired that shell 1600 resist degradation after being exposed to a target tissue site. For example, PGA may degrade in a short time (e.g., a week or so), PLA may degrade in terms of years (e.g., a year and a half), and PLGA and PGLA may degrade at a rate between that of PGA and PLA.

A cross-sectional shape of shell 1600 along an axis perpendicular to the lengthwise axis may be circular, spherical, hemispherical, square, rectangular, polygonal, or irregular, and may vary along a length of shell 1600. Shell 1600 defines a cavity 1620.

In the embodiment illustrated in FIG. 16, shell 1600 includes a pointed end 1610 integral to a remainder of shell 1600; in another embodiment (not shown), shell 1600 omits pointed end 1610, or shell 1600 and pointed end 1610 are formed separately and attached together. Pointed end 1610 may include a sharp tip (not shown) such as described and illustrated with respect to FIG. 7A or FIG. 7B. Shell 1600 may include one or more delivery assist mechanisms (not shown) such as described and illustrated with respect to FIG. 8A, FIG. 8B, FIG. 9A, or FIG. 9B, which is formed integrally with shell 1600 or attached to shell 1600.

FIG. 17A illustrates an embodiment of a plug 1710 which is disposed in cavity 1620 of shell 1600 of FIG. 16. A consistency of plug 1710 is sufficiently malleable to shape itself, or to be shaped, such that plug 1710 approximately takes a shape of cavity 1620 near pointed end 1610. In an embodiment, plug 1710 is silicone with calcium peroxide, where the components cross-linked to form the silicone are selected for a desired property of the silicone such as consistency, water transfer rate, or oxygen transfer rate. In an embodiment, plug 1710 is formed by extruding a mixture of silicone and calcium peroxide powder.

FIG. 17B illustrates an embodiment of a plug 1720 which is disposed in cavity 1620 of shell 1600 of FIG. 16. A consistency of plug 1720 is sufficiently firm such that plug 1720 approximately retains its shape when disposed in cavity 1620, which may (or may not) result in a space 1730 between plug 1720 and shell 1600. In an embodiment, plug 1720 is silicone with calcium peroxide, where the components cross-linked to form the silicone are selected for a desired property of the silicone such as consistency, water transfer rate or oxygen transfer rate. FIG. 17A and FIG. 17B illustrate plug 1720 for convenience; it is to be understood that additional or other plugs such as other examples in the present disclosure may be included, or substituted for plug 1720. In an embodiment, plug 1720 is formed by extruding a mixture of silicone and calcium peroxide powder.

FIG. 18 illustrates a membrane tube 1810. In the embodiment shown, membrane tube 1810 is sprayed with a bio-ghosting material 1820 applied through a nozzle 1830. In an embodiment, membrane tube 1810 is first plasma treated to prepare for bio-ghosting. Membrane tube 1810 may be rotated (e.g., in the direction shown by arrow 1840 or in the opposite direction), or tumbled, such that bio-ghosting material 1820 covers at least an outer surface of membrane tube 1810. Membrane tube 1810 may be closed at one end or both ends (e.g., at end 1811 and/or at end 1812). If closed at an end, pores in the end may be sized to allow passage of water and oxygen through the end.

FIG. 19 illustrates an embodiment in which membrane tube 1810 is placed into cavity 1620 of shell 1600 of FIG. 17B, adjacent to plug 1720.

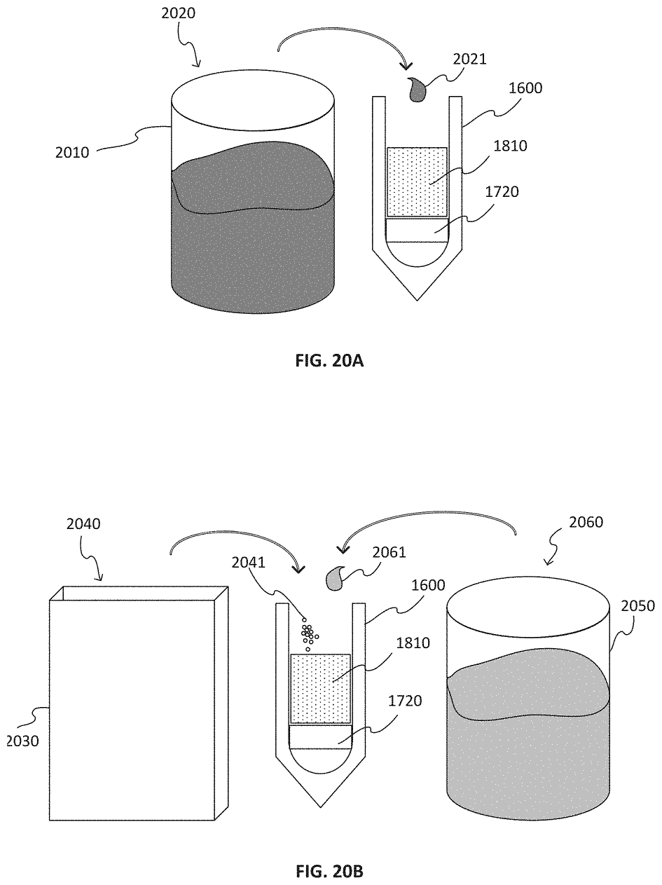

FIG. 20A illustrates an embodiment of method in which a vessel 2010 is provided, containing a gel medium 2020 in which cells are suspended. Drops 2021 of gel medium 2020 are placed (e.g., poured, dripped, spooned, or pipetted) into membrane tube 1810 within shell 1600 of FIG. 16.

In another embodiment (not shown) with respect to FIG. 20A, cells are not suspended in gel medium 2020, and cells are added to drops 2021 of gel medium 2020 after (or concurrently with) drops 2021 being placed in membrane tube 1810.

FIG. 20B illustrates an embodiment of a method in which a container 2030 is provided, containing a hardener 2040 in powdered form (in another embodiment, hardener 2040 is in liquid form). Particles 2041 of hardener 2040 (or drops of hardener 2040) are placed (e.g., poured, dripped, spooned, or pipetted) into membrane tube 1810 within shell 1600. Also provided is a vessel 2050 containing a liquid medium 2060 (e.g., an alginate) in which cells are included. Drops 2061 of liquid medium 2060 are placed (e.g., poured, dripped, spooned, or pipetted) into membrane tube 1810 within shell 1600 either before, concurrently with, or after particles 2041. Particles 2041 and drops 2061 mix within membrane tube 1810 and cross-link to form a gel.

In another embodiment (not shown) with respect to FIG. 20B, cells are not included in liquid medium 2060, and cells are added to drops 2061 of liquid medium 2060 after (or concurrently with) drops 2061 being placed in membrane tube 1810.

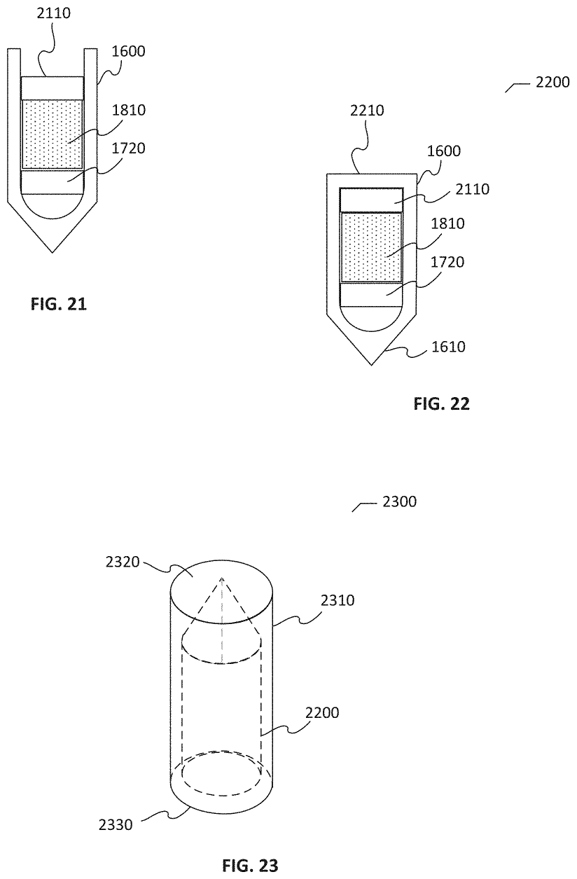

FIG. 21 illustrates an embodiment in which a second plug 2110 is disposed over membrane tube 1810, such that membrane tube 1810 has plug 1720 at one end and plug 2110 at the other end. Plug 2110 may be of similar design to plug 1720, or may be a different design. For example, an oxygen supply component in plug 2110 may be different than an oxygen supply component in plug 1720, or components cross-linked to form a framing portion (e.g., silicone) of plug 2110 may be different or have different relative percent weights than components cross-linked to form a framing portion of plug 1720.

FIG. 22 illustrates an embodiment of a cell delivery article 2200 including shell 1600 sealed to form a sealed end 2210, to fully enclose plug 1720, plug 2110, and membrane tube 1810.

In an embodiment, shell 1600 is designed to withstand degradation for a time after delivery to a tissue site (e.g., days, weeks, months, or years). In this embodiment, shell 1600 is porous (in addition to membrane tube 1810 being porous), and is coated with a bio-ghost material (in addition to or alternatively to membrane tube 1810 being coated with a bio-ghost material). Pores are sized to allow oxygen, nutrients, other cell factors, interstitial fluid, and so forth to pass into shell 1600, and to allow cell products to pass out of shell 1600, while blocking immune cells and proteins from entering shell 1600. The bio-ghost coating minimizes or prevents immune system attacks and fibrotic development from occurring on portions of a surface of shell 1600 where the bio-ghost coating is disposed.

FIG. 20A, FIG. 20B, FIG. 21, and FIG. 22 illustrate plug 1720 for convenience; it is to be understood that additional plugs such as other examples in the present disclosure may be added, or substituted for plug 1720. In an embodiment, plug 1720 is formed by extruding a mixture of silicone and calcium peroxide powder.

FIG. 23 illustrates in perspective view an embodiment of a sealed chamber 2300 including a chamber cylinder 2310 in which cell delivery article 2200 of FIG. 22 is disposed. Chamber cylinder 2310 is sealed on one end with a seal 2320 (e.g., aluminum foil) and on the opposite end with a seal 2330 (e.g., aluminum foil) Chamber 2300 may be used to contain cell delivery article 2200 until it is delivered, or until it is formulated into a composition and dosage form. In an embodiment, cell delivery article 2200 is manufactured in an aseptic environment, and cell delivery article 2200 is sealed into chamber 2300 in the aseptic environment, so that cell delivery article 2200 remains in an aseptic space in chamber 2300 until chamber 2300 is breached (e.g., seal 2320 or seal 2330 is pierced or removed). Although chamber 2300 is illustrated as including a single cell delivery article 2200, a chamber similar to chamber 2300 may contain multiple cell delivery articles.

In an embodiment, a sealed chamber (e.g., chamber 2300) is emptied of the cell delivery article(s) (e.g., cell delivery article 2200) to prepare a composition and dosage form. For example, multiple cell delivery articles may be emptied from one or more chambers into a liquid, suspension, or paste to form a composition which is then prepared in a suitable dosage form. Compositions and dosage forms are discussed elsewhere in the present disclosure.

In an embodiment, a sealed chamber (e.g., chamber 2300) retains the cell delivery article(s) (e.g., cell delivery article 2200) in an aseptic space within the sealed chamber until the cell delivery articles(s) are ejected from the sealed chamber into a subject body. For example, a tool may be used to pierce seal 2330 of chamber 2300 and push against sealed end 2210 of cell delivery article 2200, thereby causing pointed end 1610 of cell delivery article 2200 to pierce seal 2320 of chamber 2300 and enter the subject body. Such a tool may be hand-held (e.g., for some embodiments of parenteral delivery) or may be self-actuating (e.g., for oral delivery or timed delivery).

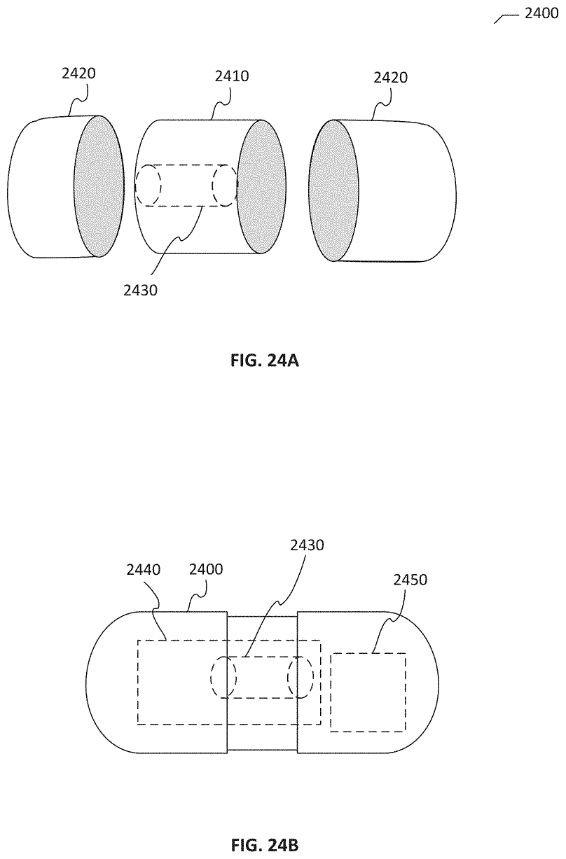

FIG. 24A illustrates in perspective view components of an embodiment of a capsule 2400. In this embodiment, capsule 2400 is assembled from a cylindrical segment 2410 and two end caps 2420. A chamber 2430 (e.g., similar to chamber 2300) is disposed within cylindrical segment 2410, and end caps 2420 are placed onto and affixed to (e.g., by adhesion, friction, or compression) cylindrical segment 2410. At least portions of capsule 2400 are structured to degrade under conditions near a target tissue site. For example, cylindrical segment 2410 may be structured to quickly (e.g., within seconds or minutes) degrade upon exposure to the conditions near the target tissue site, to expose contents of capsule 2400 to biological matter at the target tissue site; end caps 2420 may also be structured to quickly degrade, or may be structured to degrade more slowly than cylindrical segment 2410 or to not substantially degrade until after removal or expunging from the body. For another example, end caps 2420 may be structured to degrade before, significantly before, or in approximately a same time frame, as cylindrical segment 2410.

FIG. 24B illustrates in side view an embodiment of assembled capsule 2400 containing chamber 2430 and also containing a self-actuating tool 2440 and/or a self-actuating tool 2450.

In an embodiment, capsule 2400 includes self-actuating tool 2440 and omits self-actuating tool 2450. In this embodiment, chamber 2430 is contained within self-actuating tool 2440, and after at least portions of capsule 2400 degrade, self-actuating tool 2440 actuates to forcibly expel the cell delivery article(s) from chamber 2430 and from self-actuating tool 2440. Forcible expulsion may be caused, for example, by a spring force, by an explosive force, or by a buildup of pressure.

In an embodiment, capsule 2400 includes self-actuating tool 2450 and omits self-actuating tool 2440. In this embodiment, chamber 2430 is adjacent to self-actuating tool 2450, and after at least portions of capsule 2400 degrade, self-actuating tool 2450 actuates to forcibly expel the cell delivery article(s) from chamber 2430. Forcible expulsion may be caused, for example, by a spring force, by an explosive force, or by a buildup of pressure.

In an embodiment, capsule 2400 includes self-actuating tool 2440 and self-actuating tool 2450. Chamber 2430 is contained within self-actuating tool 2440, and self-actuating tool 2440 is adjacent to self-actuating tool 2450. After at least portions of capsule 2400 degrade, self-actuating tool 2440 and self-actuating tool 2450 actuate in concert or in sequence to forcibly expel the cell delivery article(s) from chamber 2430. Forcible expulsion may be caused, for example, by a spring force, by an explosive force, or by a buildup of pressure.

In an embodiment, capsule 2400 does not include self-actuating tool 2440 or self-actuating tool 2450. After at least portions of capsule 2400 degrade, chamber 2430 at least partially degrades to expose cell delivery article 2430 to the tissue site.

### Example 2

A subject with diabetes mellitus may be treated by orally administering a cell delivery article (e.g., cell delivery article 2430 in capsule 2400). The subject's treatment plan may include delivery of about 200,000 pancreatic islets, with more islet delivered if needed. In an embodiment, each cell delivery article contains about 300 islets, and each capsule contains 2 cell delivery articles; a dosing schedule includes taking 1 capsule by mouth 2 times per day for 4 months, with monitoring of indicators of pancreatic health (e.g., glycemic control) during the treatment period to determine if the dosing schedule should be extended or modified.

In this example, the cell delivery articles represent tiny pancreases, such that there is effectively a mini-organ transplant each day. The cell delivery articles incorporate into a tissue site and cells in the cell delivery articles express cell products to the subject body for an extended period of time (e.g., months or years). Thus, each daily oral administration augments a capability of the subject body's pancreas (if still functioning), and all of the mini-organ transplants that have been previously administered and are still functioning, to express cell products to maintain glycemic control. For example, over time, dozens or hundreds or thousands of mini-organs may be spread throughout an organ, or throughout a subject's body.

In this and other examples for treatment of other conditions, various ones of the mini-organ may contain different cell types for treatment of multiple conditions, or treatment of a condition in multiple ways.

### Example 3

In an embodiment, for a composition and dosage form suitable for liquid injection, a coating over a cell delivery article is designed to dissolve at an intended tissue site and not dissolve within the liquid dosage form.

### Example 4

In an embodiment, for a composition and dosage form using a slurry, a coating over a cell delivery article is designed to dissolve at an intended tissue site and not dissolve within the slurry.

### Example 5

In an embodiment, for a treatment into the brain, multiple cell delivery articles are delivered into the brain (e.g., into cerebrospinal fluid or into or adjacent to a tumor), through a catheter.

### Example 6

In an embodiment, a dosage form includes a transporter in the form of a balloon. Multiple cell delivery articles are attached to the balloon with a material such as a sugar that dissolves quickly when in contact with a liquid. The balloon is then expanded into a space such that the balloon presses into tissue within the space and the cell delivery articles are forced into the tissue. For example, a balloon may be used in the esophagus, stomach, a vein, an artery, a lung, the heart, the intestine, the brain, and other space.

### Example 7

In an embodiment, one or more cell delivery articles are implanted in the eye.

### Example 8

In an embodiment, a cell delivery article is structured such that all cells contained in a reservoir in the cell delivery article are no more about 100 μm distant from a plug in the cell delivery article. In an embodiment, a cell delivery article includes a plug positioned lengthwise along a longest axis in a reservoir (see, e.g., plug 410, 410a, 410b, 410c respectively in FIG. 4A-FIG. 4D). The plug is structured such that a distance from the plug to an outer wall of the reservoir (e.g., such as a reservoir outer wall that may be defined by outer perimeter 401 in FIG. 4A-4D) does not exceed about 100 μm. In an embodiment, the cell delivery article has a length of up to about 25 cm, and a cross-sectional dimension of up to about 1000 μm.

For example, a cell delivery article is cylindrical with a length of approximately 15 cm and a diameter of about 1000 μm, and a plug having a diameter of about 800 μm extends along a length of a cavity defined by the cell delivery article.

The cell delivery articles of Example 8 may be positioned within a peritoneal cavity of a subject or other cavity of the subject.

### Example 9

In an embodiment, a cell delivery article is structured such that all cells contained in a reservoir in the cell delivery article are no more than about 500 μm distant from a plug in the cell delivery article, such as no more than about 100 μm, no more than about 200 μm, no more than about 300 μm, no more than about 400 μm, or no more than about 500 μm.

### Example 10

In an embodiment, a cell delivery article is structured with a plug formed in a helical fashion disposed in a reservoir of the cell delivery article.

### Example 11

In an embodiment, a cell delivery article includes a reservoir having a length and a cross-sectional dimension (e.g., diameter or width). A ratio of the length to the cross-sectional dimension is greater than 2:1.

### CONCLUSION

Embodiments include without limitation the following:
In an aspect, a cell delivery article includes a reservoir, at least one plug in chemical communication with the reservoir, and cells disposed within the reservoir. The reservoir includes a reservoir outer wall, and the plug includes an oxygen supply component.
In an aspect, a cell delivery article includes a reservoir, one or more plugs, a medium disposed within the reservoir and structured to support the cells, and a bio-ghost coating covering at least a portion of an outer wall of the reservoir. Each plug is in chemical communication with the reservoir, and includes an oxygen supply component.
In an aspect, a method of delivering cells to a tissue site includes introducing a cell delivery article into a body of a subject. The cell delivery article includes cells within a reservoir, a medium disposed within the reservoir and structured to support the cells, and an oxygen supply. The reservoir includes a porous outer wall. The cell delivery article further includes a bio-ghost coating covering at least a portion of the reservoir outer wall. The bio-ghost coating may include a biomimetic peptide. The method further includes generating oxygen from the oxygen supply to assist in the support of the cells, and preventing recognition of the cell delivery article by the immune system of the subject using the bio-ghost coating.
In an aspect, a method of administering cells to a subject includes providing a treatment regimen for a condition, the treatment regimen including a dosing schedule, and administering a dose including one or more cell delivery articles in a dosage form according to the dosing schedule. Each cell delivery article includes multiple cells, a medium, and an oxygen supply for supporting the cells.

An embodiment of any of the foregoing aspects may include one, or a combination of, the following features:
A plug includes silicone.
An oxygen supply component includes one or more formulations selected from a group including: calcium peroxide, sodium peroxide, and magnesium oxide.
The cell delivery article includes a delivery assist mechanism structured to assist in delivery of the article to, or retention of the article in, tissue.
The reservoir outer wall defines pores sized to allow passage of oxygen and nutrients, and prevent passage of immune system cells and proteins.
Each of the pores of a reservoir outer wall has a diameter falling within a range between 0.2 μm and 7 μm.
A cell delivery article includes a bio-ghost coating covering at least a portion of the reservoir outer wall, wherein the bio-ghost coating is structured to prevent triggering of the immune response.
A bio-ghost coating includes a biomimetic peptide. An example is a multi-arm peptide, which may be an analogue of the cell binding domain of collagen.
The reservoir outer wall includes polytetrafluoroethylene (PTFE), expanded polytetrafluoroethylene (ePTFE), porous polyimide, polysulfone, and/or cellulose.
The reservoir outer wall includes a membrane of porous polyimide.
The cell delivery article includes a medium structured to support the cells. The medium includes alginate, alginate gel, polylysine, poly-L-ornithine, agarose, polyethylene glycol, chitosan, collagen, polydiallydimethyl ammonium chloride, or combinations thereof.
The cells include a pancreatic islet, or an alpha, beta, or delta islet cell, or a combination of alpha, beta and/or delta islet cells.
The cells produce insulin, glucagon, or a combination thereof.
The cells include an incretin cell. The incretin cell may be a K cell, an L cell, or a combination thereof.
The cells produce gastric inhibitory peptide, glucagon-like peptide, or a combination thereof.
The cells include an immune cell. The immune cell may be a T-cell, a B-cell, an NK cell, a macrophage, a neutrophil, or a genetically modified variant of one of the foregoing. A macrophage may produce TNF-alpha.
The cells include a stem cell. The stem cell may be an embryonic stem cell, an endothelial progenitor cell, a hematopoietic stem cell, a mesenchymal stem cell, a neural stem cell, a keratinocyte stem cell, or a genetically modified variant of one of the foregoing.
The cells include a chondrocyte and/or a fibroblast.
The cells produce parathyroid hormone and/or thyroid hormone.
The cells produce estrogen, progesterone, testosterone, or a combination thereof.
The cells produce growth hormone.
The cell delivery article may be delivered orally, intravenously, intramuscularly, subcutaneously, topically, intraperitoneal, or by any other mode of delivery.
The cell delivery article may be delivered to a tissue site in the small intestine, the large intestine, the colon, the liver, the omentum, the peritoneum, an ovary, the uterus, the thyroid, the brain, the intrathecal space, skin, muscle, a blood vessel, an eye, or any other organ or tissue site in a body.
The cell delivery article may be maintained at the tissue site for a time period sufficient to allow incorporation of the cells into the tissue site. For example, the cell delivery article may be maintained at the tissue site by controlling a degradation rate of the cell delivery article. A time period for maintaining the cell delivery article at the tissue site may range from about two days to about three months, from about one month to about six months, from about three months to about one year, from about six months to about two years, or any other range.
A dosing schedule includes administering a dose periodically. The dosing schedule may be determined based on a subject's personalized needs. The dosing schedule may be, for example, once a day for a predetermined number of days, once a week for a predetermined number of weeks, once a month for a predetermined number of months, multiple doses in a day, and so forth. The dosing schedule includes administering the dose.
A dosage form may be a liquid, a pill, a tablet, a soft-gel, a film, a patch, a cream, gel, an ointment, or any other applicable dosage form. A dosage form may include a transporter. For example, a dosage form may include a capsule.
A condition to be treated may be, for example, diabetes, pancreatitis, cancer, thyroid disease, growth deficiency, neurological disease, a burn or a wound.
The cells included in a cell delivery article are pancreatic islet cells, and a treatment regimen includes evaluating one or more indicators of pancreatic health of the subject, and either sustaining the treatment regimen if the evaluation does not indicate pancreatic health, or revising the treatment regimen if the evaluation does indicate pancreatic health.
Other features as described in the present disclosure.

While the present disclosure has been described and illustrated with reference to specific embodiments thereof, these descriptions and illustrations do not limit the present disclosure. It can be clearly understood that various changes can be made, and equivalent components can be substituted within the embodiments, without departing from the true spirit and scope of the present disclosure as defined by the appended claims. Also, components, characteristics, or acts from one embodiment can be readily recombined or substituted with one or more components, characteristics or acts from other embodiments to form numerous additional embodiments within the scope of the invention. Moreover, components that are shown or described as being combined with other components, can, in various embodiments, exist as standalone components. Further, for any positive recitation of a component, characteristic, constituent, feature, step or the like, embodiments of the invention specifically contemplate the exclusion of that component, value, characteristic, constituent, feature, step or the like. The illustrations may not necessarily be drawn to scale. There can be distinctions between the artistic renditions in the present disclosure and the actual apparatus, due to variables in manufacturing processes and such. There can be other embodiments of the present disclosure which are not specifically illustrated. The specification and drawings are to be regarded as illustrative rather than restrictive. Modifications can be made to adapt a particular situation, material, composition of matter, method, or process to the objective, spirit and scope of the present disclosure. All such modifications are intended to be within the scope of the claims appended hereto. While the methods disclosed herein have been described with reference to particular operations performed in a particular order, it can be understood that these operations can be combined, sub-divided, or re-ordered to form an equivalent method without departing from the teachings of the present disclosure. Therefore, unless specifically indicated herein, the order and grouping of the operations are not limitations of the present disclosure.

1. A cell delivery article, comprising:
a reservoir, the reservoir comprising a reservoir outer wall:
at least one plug in chemical communication with the reservoir, the plug comprising an oxygen supply component; and
a plurality of cells disposed within the reservoir.

2. The cell delivery article of claim 1, wherein the reservoir outer wall defines a plurality of pores sized to allow passage of oxygen and nutrients, and prevent passage of immune system cells and proteins.

3. The cell delivery article of claim 2, wherein each of the pores has a diameter falling within a range between 0.2 micrometers and 7 micrometers.

4. The cell delivery article of claim 1, further comprising a bio-ghost coating covering at least a portion of the reservoir outer wall, wherein the bio-ghost coating is structured to prevent triggering of an immune response.

5. The cell delivery article of claim 4, wherein the bio-ghost coating comprises a biomimetic peptide.

6. The cell delivery article of claim 5, wherein the biomimetic peptide comprises a multi-arm peptide.

7. The cell delivery article of claim 5, wherein the multi-arm peptide is an analogue of the cell binding domain of collagen.

8. The cell delivery article of claim 1, wherein the reservoir outer wall comprises polytetrafluoroethylene (PTFE), expanded polytetrafluoroethylene (ePTFE), porous polyimide, polysulfone, or cellulose.

9. The cell delivery article of claim 1, further comprising a medium disposed within the reservoir, the medium structured to support the cells, wherein the medium comprises alginate, alginate gel, polylysine, poly-L-ornithine, agarose, polyethylene glycol, chitosan, collagen, polydiallydimethyl ammonium chloride, or a combination thereof.

10. The cell delivery article of claim 1, wherein the cells comprise a pancreatic islet, a pancreatic islet alpha cell, a pancreatic islet beta cell, a pancreatic islet delta cell, an incretin cell, an immune cell, or a stem cell.

11. The cell delivery article of claim 1, wherein the cells produce insulin, glucagon, gastric inhibitory peptide, glucagon-like peptide, parathyroid hormone, thyroid hormone, estrogen, progesterone, testosterone, growth hormone, or a combination of two or more of the foregoing.

12. The cell delivery article of claim 1, wherein the cells comprise a chondrocyte, a fibroblast, or a combination thereof.

13. The cell delivery article of claim 1, wherein one of the at least one plug comprises silicone, and the oxygen supply component comprises one or more formulations selected from a group comprising: calcium peroxide, sodium peroxide, and magnesium oxide.

14. A method of delivering cells to a tissue site comprising:
introducing a cell delivery article into a body of a subject, the cell delivery article comprising:
a plurality of cells within a reservoir, the reservoir comprising a porous outer wall;
a medium disposed within the reservoir, the medium structured to support the plurality of cells;
an oxygen supply; and
a bio-ghost coating covering the reservoir outer wall, the bio-ghost coating comprising a biomimetic material;
generating oxygen from the oxygen supply to assist in the support of the plurality of cells; and
preventing recognition of the cell delivery article by the immune system of the subject using the bio-ghost coating.

15. The method of claim 14, wherein the bio-ghost coating comprises a multi-arm peptide that is an analogue of the cell binding domain of collagen.

16. The method of claim 14, wherein the tissue site is the small intestine, the large intestine, the colon, the liver, the omentum, the peritoneum, an ovary, the uterus, the thyroid, the brain, the intrathecal space, skin, muscle, a blood vessel, or an eye.

17. The method of claim 14, further comprising maintaining the cell delivery article at the tissue site for a time period sufficient to allow incorporation of the cells into the tissue site.

18. A method of administering cells to a subject comprising:
providing a treatment regimen for a condition comprising a dosing schedule; and
administering a dose comprising one or more cell delivery articles in a dosage form according to the dosing schedule,
wherein the one or more cell delivery articles comprise a plurality of cells, and a medium and an oxygen supply for supporting the cells.

19. The method of claim 18, wherein the dosing schedule comprises administering the dose periodically.

20. The method of claim 18, wherein the dosing schedule comprises administering multiple doses once a day for a predetermined number of days, once a week for a predetermined number of weeks, or once a month for a predetermined number of months.

21. The method of claim 18, wherein the dosage form is a liquid, a pill, a tablet, a capsule, a soft-gel, a film, a patch, a cream, a gel, or an ointment.

22. The method of claim 18, wherein the plurality of cells comprises pancreatic islet cells, stem cells, incretin cells, immune cells, fibroblasts, chondrocytes, or combinations thereof.

23. The method of claim 18, wherein the condition is selected from the group consisting of diabetes, pancreatitis, cancer, thyroid disease, growth deficiency, and neurological disease.

24. The method of claim 18, wherein the condition is a burn or a wound.

25. The method of claim 18, wherein the cells comprise pancreatic islet cells, and the treatment regimen further comprises:
evaluating one or more indicators of pancreatic health of the subject; and
either sustaining the treatment regimen if the evaluation does not indicate pancreatic health, or revising the treatment regimen if the evaluation does indicate pancreatic health.
